(19) United States
(12) Patent Application Publication
(10) Pub. No.: US 2015/0087045 A1
(43) Pub. Date: Mar. 26, 2015

(54) CRYSTAL STRUCTURE OF HCV POLYMERASE COMPLEXES AND METHODS OF USE

(71) Applicant: GILEAD PHARMASSSET LLC, FOSTER CITY, CA (US)

(72) Inventors: Thomas E. Edwards, Bainbridge Island, WA (US); Angela Man Iu Lam, Fort Lee, NJ (US); Ralph T. Mosley, Roselle, NJ (US); Eisuke Murakami, Perth Amboy, NJ (US)

(21) Appl. No.: 14/371,846

(22) PCT Filed: Jan. 11, 2013
(86) PCT No.: PCT/US2013/021130

§ 371 Date: Jul. 11, 2014

Related U.S. Application Data
(60) Provisional application No. 61/586,584, filed on Jan. 13, 2012.

(51) Int. Cl. C12N 9/12; C12N 7/00; C12N 9/96

(52) U.S. Cl. CPC C12N 9/127; C12N 9/96; C12N 7/00; C12N 2770/24222; C07K 2299/00
USPC 435/188; 536/23.2; 435/194

(57) ABSTRACT

The present disclosure includes a crystalline form and a crystal structure of HCV RNA polymerase and HCV RNA polymerase in a complex with an RNA template primer molecule. In other aspects, the disclosure provides methods of using the crystal structures and structural coordinates to identify homologous proteins and to design or identify agents that can modulate the function of the HCV RNA polymerase and HCV RNA polymerase in a complex with an RNA template primer molecule.

### CROSS-REFERENCE TO RELATED APPLICATIONS

This application claims the benefit of priority from U.S. Provisional Patent Application No. 61/586,584, filed Jan. 13, 2012, the content of which is hereby incorporated by reference herein in its entirety.

### BACKGROUND OF THE INVENTION

### Field of the Invention

An estimated 180 million people worldwide are infected with the hepatitis C virus (HCV). Approximately 80% of these will develop chronic liver disease, and a significant subset will progress to cirrhosis of the liver and eventually death (Lavanchy (2009) Liver Int. 29(Suppl. 1):74-81). HCV is a small, single-stranded positive-sense RNA virus and, like dengue virus, bovine viral diarrhea virus, and West Nile virus, is a member of the Flaviviridae family of viruses. The nonstructural 5B (NS5B) protein, a 66 kDa protein of ˜590 amino acids found at the C-terminus of the virally encoded HCV polyprotein, provides the requisite RNA-dependent RNA polymerase (RdRp) functionality (Penin et al. (2004) Hepatology 39:5-19). The polymerase produces positive RNA strands for encapsidation into viral particles by using an intermediate negative RNA strand that it synthesizes from the initial positive strand RNA template provided by the virus. GTP-dependent de novo initiation is the preferred mode of nucleotide polymerization in vivo.⁷

Nearly one hundred crystal structures of HCV NS5B have been reported covering genotypes 1a, 1b, 2a, and 2b, although all structures lack the C-terminal membrane anchoring tail⁴. HCV NS5B exhibits the “right hand” shape common to many polymerases, along with easily recognized “fingers,” “palm,” and “thumb” domains⁸⁻¹⁰. Extensive efforts to obtain a high resolution crystal structure of wild-type HCV polymerase in complex with growing RNA primer-template pairs have proven unsuccessful, although a structure has been reported with a polyuridine template in an unproductive conformation¹¹. Superposition of NS5B and HIV-1 reverse transcriptase¹² crystal structures provided the earliest models for HCV elongation⁸⁻¹⁰. However, a β-hairpin loop spanning residues 442-454 of the thumb domain and a C-terminal linker blocks egress necessary for elongation and, as was observed with HIV-1 RT, the thumb domain has been predicted to move in the presence of RNA^{8,9}. By analogy to a bacteriophage φ-6 polymerase initiation complex with GTP and template¹³, Tyr448 of this β-hairpin loop may stack against the initiating GTP during de novo initiation. Crystal structures provide structural information regarding mechanism of action of molecules.

Thus, there remains a need to identify new therapeutics useful to HCV infections and other diseases associated with HCV polymerase activity. A high resolution crystal structure of wild-type HCV polymerase in complex with growing RNA primer-template is useful in the design of inhibitors of HCV RNA polymerase and HCV infection.

### SUMMARY OF THE INVENTION

The present disclosure thus includes a crystalline form and a crystal structure of HCV RNA polymerase and HCV RNA polymerase in a complex with an RNA template primer molecule. In other aspects, the disclosure provides methods of using the crystal structures and structural coordinates to identify homologous proteins and to design or identify agents that can modulate the function of the HCV RNA polymerase and HCV RNA polymerase in a complex with an RNA template primer molecule. The present disclosure also includes the three-dimensional configuration of points derived from the structure coordinates of at least a portion of HCV RNA polymerase and HCV RNA polymerase in a complex with an RNA template primer molecule, as well as structurally equivalent configurations, as described herein. The three-dimensional configuration includes points derived from structure coordinates representing the locations of a plurality of the amino acids defining the HCV RNA polymerase active site when it is not bound to substrate or when it is bound to a substrate.

Likewise, the disclosure also includes the scalable three-dimensional configuration of points derived from structure coordinates of molecules or molecular complexes that are structurally homologous to HCV RNA polymerase and HCV RNA polymerase in a complex with an RNA template primer molecule as well as structurally equivalent configurations. Structurally homologous molecules or molecular complexes are defined herein. Advantageously, structurally homologous molecules can be identified using the structure coordinates of the HCV RNA polymerase and HCV RNA polymerase in a complex with an RNA template primer molecule according to a method of the disclosure.

The configurations of points in space derived from structure coordinates according to the disclosure can be visualized as, for example, a holographic image, a stereodiagram, a model, or a computer-displayed image, and the disclosure thus includes such images, diagrams or models.

The crystal structure and structural coordinates can be used in methods, for example, for obtaining structural information of a related molecule, and for identifying and designing agents that modulate HCV RNA polymerase activity.

The coordinates of HCV RNA polymerase are provided in Table 1. The coordinates of HCV RNA polymerase in a complex with an RNA template primer molecule are provided in Tables 2 and 3.

### BRIEF DESCRIPTION OF THE DRAWINGS

FIG. 1A/1B shows de novo RNA synthesis activity of HCV NS5B polymerase 1b WT, 1b S282T, and 1b Δ8 constructs. In 1A, the radioactive RNA products were separated from unreacted substrates using a Hybond N+ membrane (GE Healthcare) as described previously. The products were visualized and quantified using a phosphorimager. In 1B, the reaction rates were calculated using GraphFit (Erithacus Software, Horley, Surrey, UK). Samples 1b Δ8_{—}1 and 1b Δ8_{—}2 indicate samples from two separate preparations of 1b Δ8 protein.

FIG. 2 shows RNA synthesis and chain termination by PSI-352666 for HCV NS5B polymerase 1b Δ8 construct. The PSI-352666-terminated product (*GGCX) was not further elongated in the presence of the next correct incoming nucleotide (ATP). Lanes indicate 0, 2, 5, 10, 20, 40, 60 min time course after preincubation.

FIG. 3 shows chain termination of HCV polymerase 1b WT, 1b S282T, 1b Δ8, and 1b Δ8 S282T with PSI-352666 or 2′-C-MeGTP.

FIG. 4 shows thermofluor analysis of NS5B polymerase 1b WT in the absence and presence of symmetrical primer-template RNAs (top) or fused primer-template hairpin RNAs (bottom).

FIG. 5 shows thermofluor analysis of NS5B polymerase 1b Δ8 in the absence and presence of symmetrical primer-template RNAs (top) or fused primer-template hairpin RNAs (bottom).

FIG. 6A/6B shows thermofluor analysis of NS5B polymerase 2a WT in the presence of symmetrical primer-template RNAs (top) or fused primer-template hairpin RNAs (bottom).

FIG. 7 shows crystals of apo 2a Δ8 grown in the Hampton Index screen condition E9 (30% pentaerythritol ethoxylate, 50 mM BisTris pH 6.5, 50 mM ammonium sulfate) that produced the 2.5 Å resolution apo structure.

FIG. 8 shows crystals of apo HCV NS5B 2a Δ8 obtained from the Hampton Index screen condition G6 (0.2 M ammonium acetate, 0.1 M BisTris pH 5.5, 25% PEG 3350) that were used for soaking experiments and produced a 2.9 Å resolution structure with 5′-UACCG(3′-dG) and a 3.0 Å resolution structure with 5′-CAUGGC(2′,3′-ddC).

FIG. 9 shows electron density maps for the RNA component of crystal structures of HCV NS5B polymerase 2a Δ8 solved with 5′-UACCG(3′-dG) at 2.9 Å resolution (top) and 2a Δ8 solved with 5′-CAUGGC(2′,3′ ddC) at 3.0 Å resolution (bottom). At left, omit electron density maps |Fₒ|−|F_{c}| into which the symmetrical primer-template RNA models were built are shown in green mesh contoured at 3.0σ. At right, final refined electron density maps 2|Fₒ|−|F_{c}|. Maps are overlaid with the final refined RNA model. For each structure, the template strand is shown in salmon colored carbon backbone and the primer strand is shown in cyan colored carbon backbone.

FIG. 10a/10b shows the structure of HCV NS5B polymerase and activity of an internal deletion variant. 10a, Crystal structure of genotype 2a HCV NS5B RdRp²³ with the fingers, palm, thumb, and C-terminal linker domains numbered and colored according to convention¹⁰. The β-hairpin loop that was deleted in the current work is colored in yellow. Two loop elements extend from the finger to the thumb domain as if the HCV RdRp “right hand” is making the “OK” gesture and thus completely encircling the NTP entrance into the catalytic site¹⁰. The β-strand fingers region potentially provides access for the incoming template RNA strand whereas the α-fingers region provides part of the proposed exit route for the double stranded RNA product. The palm domain is the most well-conserved structural feature across all of the known polymerases and contains the catalytic residues. The thumb domain appears to have the most variability among the various polymerases. In HCV NS5B, it comprises ˜160 amino acids, which is significantly larger than in other polymerases, a trait of the Flaviviridae RdRps.³⁰ This region contains seven α-helices and a relatively unique β-strand that descends into the palm domain partially blocking what is undoubtedly the exit path for the RNA product strand. 10b, de novo RNA synthesis activity of genotype 2a JFH1 isolate, wild-type HCV NS5B (2a WT) and a construct in which the β-hairpin loop has been deleted and replaced with a Gly-Gly linker (2a Δ8) demonstrating >100-fold higher total activity for 2a Δ8 compared to 2a WT. Time-dependent formation of the radiolabeled products is shown in the blot. At right, the activity for both 2a WT and 2a Δ8 were measured in the presence of the nucleotide triphosphate analog inhibitor PSI-352666, which resulted in IC₅₀ value of 6.05±0.82 μM for 2a WT and 6.41±0.75 μM for 2a Δ8.

FIG. 11 (a-c) shows a comparison of apo genotype 2a HCV NS5B wild-type and Δ8 crystal structures. 11a, Overlay of a previously determined closed crystal structure of genotype 2a HCV NS5B (PDB ID 2XXD)²³ shown in gold ribbons with a 2.5 Å resolution crystal structure of genotype 2a HCV NS5B polymerase determined here colored as in FIG. 1. Residues 62-350 of the finger and palm domain were aligned to demonstrate the large movement in the thumb domain. 11b, Interactions of the thumb domain in the closed 2a WT structure²³. 11c, Interactions of the thumb domain in the open 2a Δ8 structure showing dramatic rearrangement of loop residues 397-405 that connect the primer buttress helix with the primer grip helix as well as movement of several helixes in the thumb domain. Panels b and c were overlaid as described for panel a.

FIG. 12 (a-d) shows crystal structures of 2a Δ8 HCV NS5B with primer-template RNA. 12a, Cartoon representation of the structure viewed from the template RNA entrance tunnel with coloring as in FIG. 1. The template strand is in salmon while the primer strand is colored in cyan. 12b, View from the RNA exit tunnel of the polymerase with the membrane face on top. 12c, Overlay of the crystal structures of 2a Δ8 with the symmetrical primer-template RNAs 5′-UACCG(3′-dG) and 5′-CAUGGC(2′,3′-ddC). Despite different sequences and chain-terminators, both primer-template pairs reside within the central cavity in the same general conformation. 12d, At left, omit electron density map |Fₒ|−|F_{c}| into which the symmetrical primer-template RNA 5′-UACCG(3′-dG) was built shown in green mesh contoured at 3.0σ and at right, the 2|Fₒ|−|F_{c}| electron density map shown in blue mesh contoured at 1.0σ. The primer strand is colored in cyan colored carbon backbone, while the template strand is colored in salmon colored carbon backbone.

FIG. 13 (a-d) shows primer-template recognition by HCV polymerase. 13a, The nucleobase of the pairing nucleotide (+1) stacks on top of the highly conserved Ile160 while base-pairing with the primer strand in the product, pretranslocation state. The 3′-residue of the primer strand contains the obligate chain-terminator 3′-dG, and resides in the pretranslocation state where the incoming NTP to bind after translocation would be expected. The 2′-hydroxyl of this residue hydrogen bonds with the highly conserved Asp225. The phosphate of the terminal nucleotide of the primer strand interacts with Arg158 of the fingers domain. 13b, All of the phosphates and 2′-hydroxyls of the template strand are recognized by HCV polymerase. The sugar and phosphate of the +2 residue of the template strand extend down the template entrance tunnel. Template strand phosphates interact with the backbone amide nitrogen atoms of Arg98 (+2) and Ala97 (+1) and the side chains of Arg168 (+0), Lys172 (−1), and Gln180 (−2). The 2′-hydroxyl of the pairing nucleotide (+1) is recognized by the backbone oxygen of strictly conserved Gly283, while the other 2′-hydroxyls of the template strand are recognized by the backbone oxygen of Val284 (+0), the side chain of Ser288 (−1), and possibly the backbone oxygen of Phe193 (−2). The main source of resistance to certain nucleotide analog inhibitors occurs at Ser282, directly below the terminal nucleotide of the primer strand. 13c, Primer strand phosphates make salt bridges with Arg158 (+1) of the fingers domain and Arg386, Arg394 (+0), Arg394 (−1), and His402 (−2) of the thumb domain. 13d, Overlay of a primer-template RNA-bound crystal structure of HCV polymerase colored as in FIG. 3 with a primer-template RNA-bound crystal structure of poliovirus polymerase (PDB ID 3OL6)¹⁸ colored yellow, showing similar overall primer-template-polymerase recognition despite substantially different thumb domains.

FIG. 14 shows a model of the active site of HCV RNA polymerase. The active site of the polymerase is depicted with a smoothed vdW surface in light yellow (using 1C2P¹⁰/1GX5² data). The catalytic divalent metal cations are shown as purple spheres interacting with the sidechains of Asp318, Asp319 (part of the GDD motif) and Asp220. Arg158 hangs from the fingerloop region and facilitates the formation of the phosphodiester link between the growing RNA strand and the incoming NTP. (Arg158 is shown without a surface for the sake of clarity.) Ser282, found to be important to the development of resistance to various 2′MeNTP-based inhibitors, forms part of the surface against which the incoming NTP must fit when being incorporated into the RNA chain. Tyr448 found in the thumb loop, may help stabilize the formation of the initiation complex by interaction with the primer GTP during de novo initiation. The C-terminal loop (in gray) would necessarily be displaced upon initiation, and both it and the thumb loop displaced during elongation.

### DETAILED DESCRIPTION OF THE INVENTION

### Definitions

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Other than in the operating examples, or where otherwise indicated, all numbers expressing quantities of ingredients or reaction conditions used herein should be understood as modified in all instances by the term “about.” The term “about” when used in connection with percentages may mean±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains.

Amino acids are represented by either “single letter” symbol or “three letter” symbol.

The following definitions are used herein, unless otherwise described:

The term “HCV RNA polymerase,” or “RdRp” as used herein, refers, to any native (whether naturally occurring or synthetic) hepatitis C virus (HCV) polypeptide that is capable of binding to RNA molecules and synthesize RNA. The non-structural 5B (NS5B) protein, a 66 kDa protein of ˜590 amino acids found at the C-terminus of the virally encoded HCV polyprotein, provides the requisite RNA-dependent RNA polymerase (RdRp) functionality.⁶ The polymerase produces positive RNA strands for encapsidation into viral particles by using an intermediate negative RNA strand which it synthesizes from the initial positive strand RNA template provided by the virus. GTP-dependent de novo initiation is the preferred mode of nucleotide polymerization in vivo. The term “wild type HCV RNA polymerase” generally refers to a polypeptide having an amino acid sequence found in a naturally occurring HCV RNA polymerase and includes naturally occurring truncated or secreted forms, and variant forms.

The full proteome sequence for genotype 2a isolate JFH1 (2a JFH1) is found in the Uniprot database Q99IB8. The full protein sequence for genotype 2a is about 3033 amino acids. The reference sequence for the wild-type HCV RNA polymerase is concerned with about 590 amino acids at the C terminal. Sequence numbering as shown in the alignment and for the purpose of numbering of amino acid positions identified herein is based on alignment to starting sequence SMSY as shown in Table 8 below. For the genotype 2a HCV RNA polymerase, the starting amino acid 1 is found at amino acid position 2443 and the ending amino acid is found at position 3033 of the sequence shown in Q99IB8 and corresponds to amino acid position 590 as used herein. One of skill in the art can readily determine corresponding amino acid residues by reference to the alignment provided in Table 8. The sequence numbering of HCV RNA polymerase 2a genotype is according to that of SEQ ID NO:1. HCV RNA polymerases from different genotypes of HCV are known. An alignment of these sequences is shown in Table 8.

TABLE 8

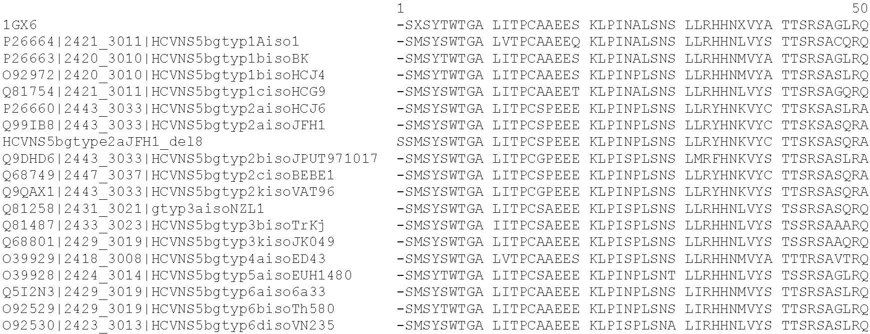

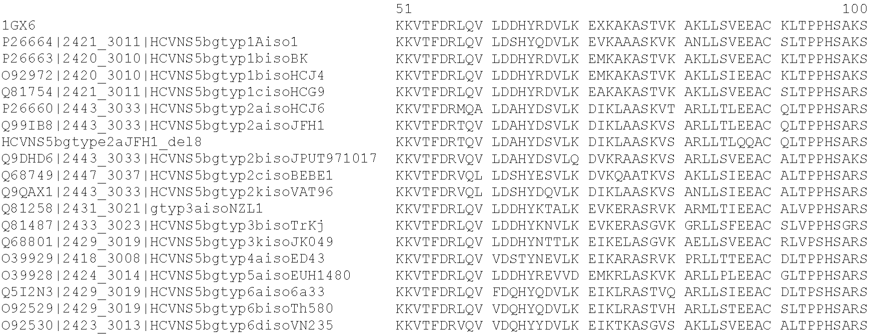

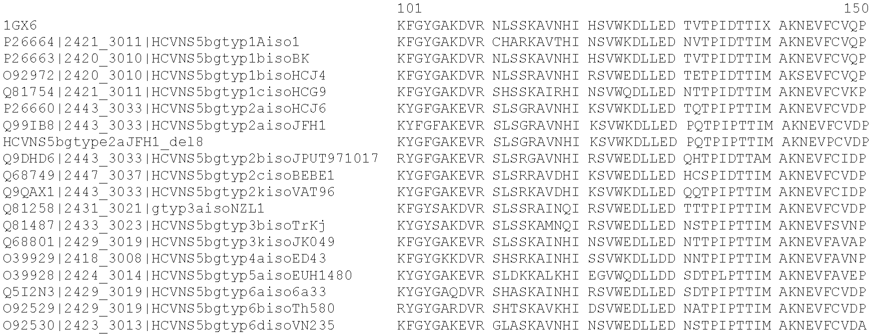

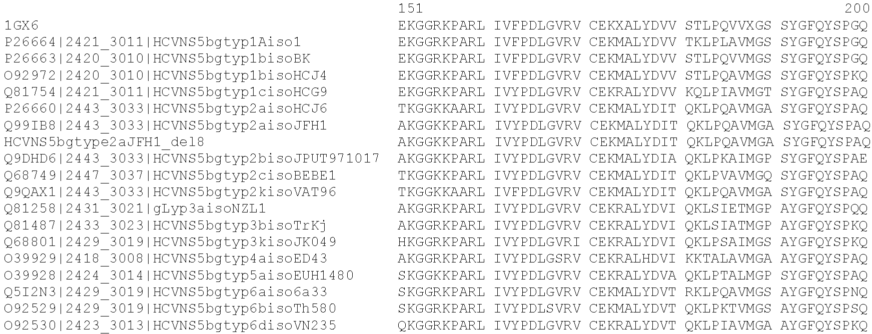

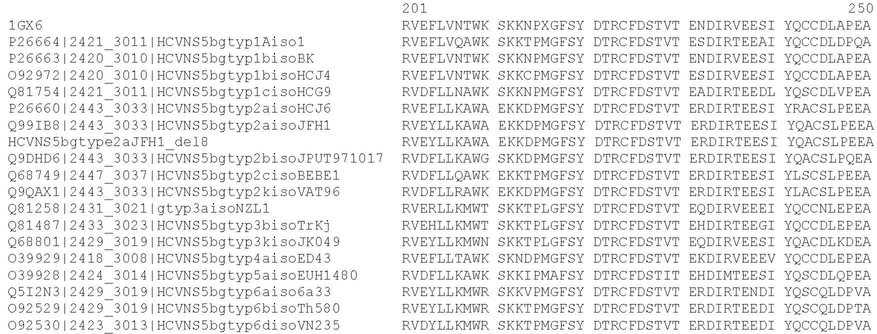

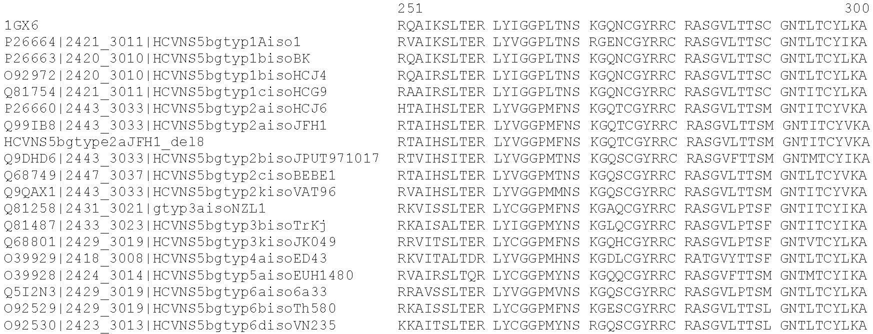

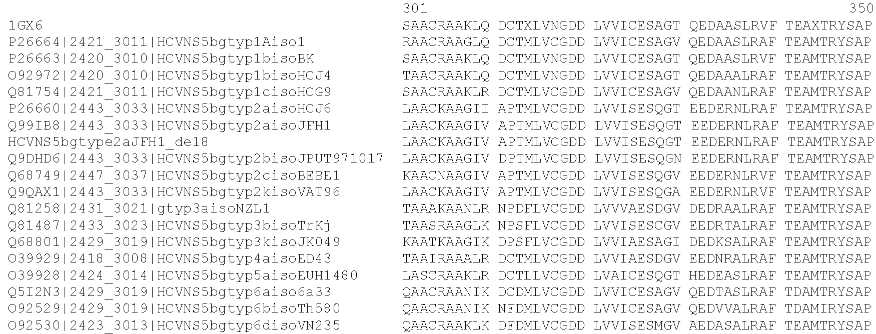

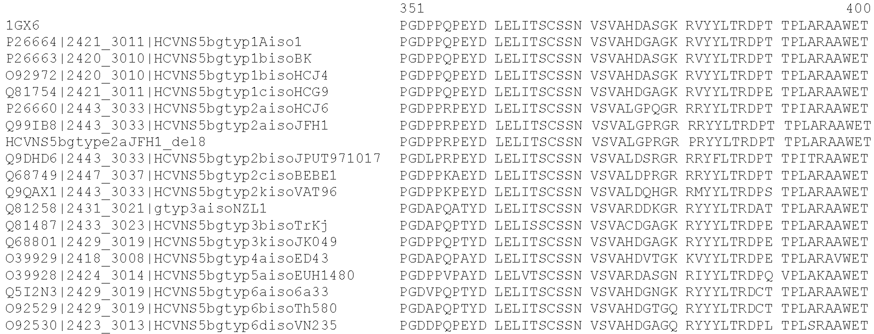

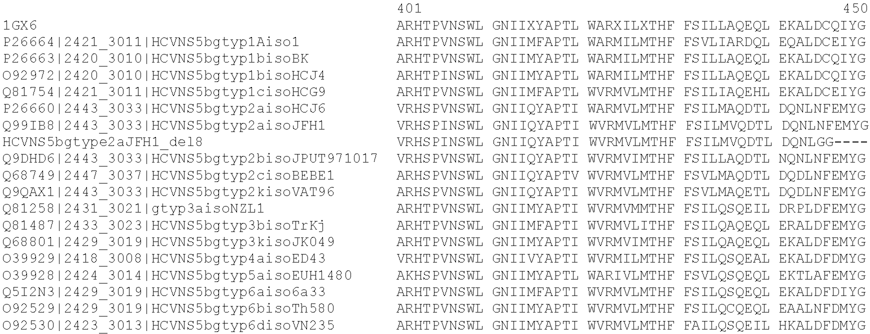

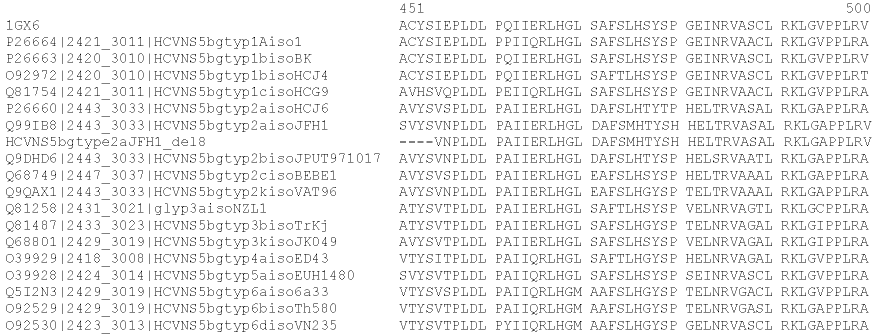

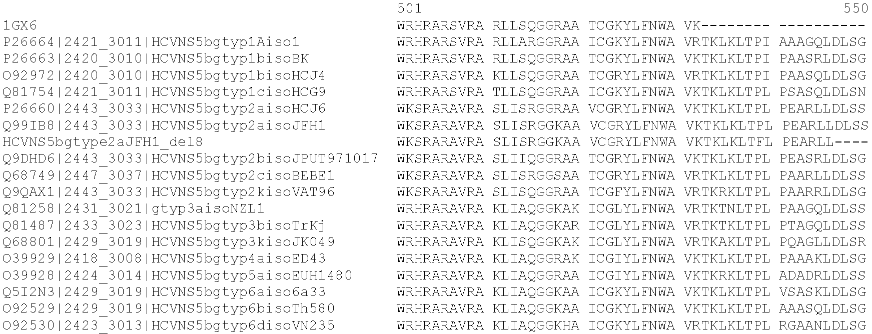

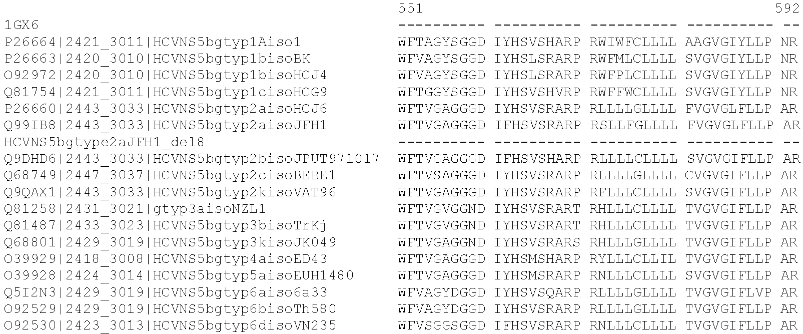

A reference sequence for the HCV RNA polymerase genotype 2a isolate JFH1 is that of SEQ ID NO:1. A reference sequence for HCV RNA polymerase 1b BK is that of SEQ ID NO:2.

>sp|Q99IB8|2443-3033
SEQ ID NO: 1
SMSYSWTGALITPCSPEEEKLPINPLSNSLLRYHNKVYCTTSKSASQRAKKVTFDRTQVL
DAHYDSVLKDIKLAASKVSARLLTLEEACQLTPPHSARSKYGFGAKEVRSLSGRAVNHIK
SVWKDLLEDPQTPIPTTIMAKNEVFCVDPAKGGKKPARLIVYPDLGVRVCEKMALYDITQ
KLPQAVMGASYGFQYSPAQRVEYLLKAWAEKKDPMGFSYDTRCFDSTVTERDIRTEESIY
QACSLPEEARTAIHSLTERLYVGGPMFNSKGQTCGYRRCRASGVLTTSMGNTITCYVKAL
AACKAAGIVAPTMLVCGDDLVVISESQGTEEDERNLRAFTEAMTRYSAPPGDPPRPEYDL
ELITSCSSNVSVALGPRGRRRYYLTRDPTTPLARAAWETVRHSPINSWLGNIIQYAPTIW
VRMVLMTHFFSILMVQDTLDQNLNFEMYGSVYSVNPLDLPAIIERLHGLDAFSMHTYSHH
ELTRVASALRKLGAPPLRVWKSRARAVRASLISRGGKAAVCGRYLFNWAVKTKLKLTPLP
EARLLDLSSWFTVGAGGGDIFHSVSRARPRSLLFGLLLLFVGVGLFLLPAR
>sp|P26663|2420-3010
SEQ ID NO: 2
SMSYTWTGALITPCAAEESKLPINALSNSLLRHHNMVYATTSRSAGLRQKKVTFDRLQVL
DDHYRDVLKEMKAKASTVKAKLLSVEEACKLTPPHSAKSKFGYGAKDVRNLSSKAVNHIH
SVWKDLLEDTVTPIDTTIMAKNEVFCVQPEKGGRKPARLIVFPDLGVRVCEKMALYDVVS
TLPQVVMGSSYGFQYSPGQRVEFLVNTWKSKKNPMGFSYDTRCFDSTVTENDIRVEESIY
QCCDLAPEARQAIKSLTERLYIGGPLTNSKGQNCGYRRCRASGVLTTSCGNTLTCYLKAS
AACRAAKLQDCTMLVNGDDLVVICESAGTQEDAASLRVFTEAMTRYSAPPGDPPQPEYDL
ELITSCSSNVSVAHDASGKRVYYLTRDPTTPLARAAWETARHTPVNSWLGNIIMYAPTLW
ARMILMTHFFSILLAQEQLEKALDCQIYGACYSIEPLDLPQIIERLHGLSAFSLHSYSPG
EINRVASCLRKLGVPPLRVWRHRARSVRARLLSQGGRAATCGKYLFNWAVKTKLKLTPIP
AASRLDLSGWFVAGYSGGDIYHSLSRARPRWFMLCLLLLSVGVGIYLLPNR
The remaining sequences presented in Table 8 are:
(SEQ ID NO: 20)
1GX6;
(SEQ ID NO: 21)
P26664|2421_3011|HCVNS5bgtyp1Aiso1;
(SEQ ID NO: 22)
O92972|2420_3010|HCVNS5bgtyp1bisoHCJ4;
(SEQ ID NO: 23)
Q81754|2421_3011|HCVNS5bgtyp1cisoHCG9;
(SEQ ID NO: 24)
P26660|2443_3033|HCVNS5bgtyp2aisoHCJ6;
(SEQ ID NO: 25)
HCVNS5bgtype2aJFH1_del8;
(SEQ ID NO: 26)
Q9DHD6|2443_3033|HCVNS5bgtyp2bisoJPUT971017;
(SEQ ID NO: 27)
Q68749|2447_3037|HCVNS5bgtyp2cisoBEBE1;
(SEQ ID NO: 28)
Q9QAX1|2443_3033|HCVNS5bgtyp2kisoVAT96;
(SEQ ID NO: 29)
Q81258|2431_3021|gtyp3aisoNZL1;
(SEQ ID NO: 30)
Q81487|2433_3023|HCVNS5bgtyp3bisoTrKj;
(SEQ ID NO: 31)
Q68801|2429_3019|HCVNS5bgtyp3kisoJK049;
(SEQ ID NO: 32)
O39929|2418_3008|HCVNS5bgtyp4aisoED43;
(SEQ ID NO: 33)
O39928|2424_3014|HCVNS5bgtyp5aisoEUH1480;
(SEQ ID NO: 34)
Q5I2N3|2_29_3019|HCVNS5bgtyp6aiso6a33;
(SEQ ID NO: 35)
O92529|2429_3019|HCVNS5bgtyp6bisoTh580;
and
(SEQ ID NO: 36)
O92530|2423_3013|HCVNS5bgtyp6disoVN235.

“HCV RNA polymerase variant” as used herein refers to polypeptide that has a different sequence than a reference polypeptide. In some embodiments, the reference polypeptide is an HCV RNA polymerase comprising SEQ ID NO:1 or 2. Variants include “non-naturally” occurring variants. In some embodiments, a variant has at least 80% amino acid sequence identity with the amino acid sequence of SEQ ID NO:1 or 2. The variants include those polypeptides that have substitutions, additions or deletions. In some embodiments, the HCV RNA polymerase has a deletion of 0 hairpin residues that correspond to amino acids 444-453 of HCV RNA polymerase genotype 2a of SEQ ID NO:3. In other embodiments, a variant HCV RNA polymerase lacks a C terminal peptide membrane binding region, such as a deletion of at least 20 to 30 amino acids. In some embodiments, a variant has one or more amino acid substitutions such as those that enhance solubility of the molecule. In some embodiments, at least one amino acid substitution is selected from the group consisting of an amino acid position corresponding to position 47, 86, 87, 114 of the HCV RNA polymerase having the amino acid sequence of SEQ ID NO:1, 2, 3, or 4, and mixtures thereof. In some embodiments, a variant HCV RNA polymerase has an amino acid substitution at an amino acid position associated with resistance to inhibitors of RNA polymerase activity. In some embodiments, a variant HCV RNA polymerase has an amino acid substitution at an amino acid position 282. In some embodiments, a variant HCV RNA polymerase has an amino acid substitution at an amino acid position selected from the group consisting of 15, 223, 321, and mixtures thereof. In some embodiments, a variant HCV RNA polymerase has an amino acid substitution in one or more of the active site residues. In some embodiments, a variant HCV RNA polymerase has an amino acid substitution at an amino acid position selected from the group consisting of Tyr448, Asp318, Asp319, Asp220, Thr221, Arg158, Asp225, Ile160, and Ser282 and mixtures thereof. In some embodiments, the variants have increased RNA polymerase activity as compared the biological activity of wild type HCV RNA polymerase.

Ordinarily, an HCV RNA polymerase variant polypeptide will have at least 80% sequence identity, more preferably will have at least 81% sequence identity, more preferably will have at least 82% sequence identity, more preferably will have at least 83% sequence identity, more preferably will have at least 84% sequence identity; more preferably will have at least 85% sequence identity, more preferably will have at least 86% sequence identity, more preferably will have at least 87% sequence identity, more preferably will have at least 88% sequence identity, more preferably will have at least 89% sequence identity, more preferably will have at least 90% sequence identity, more preferably will have at least 91% sequence identity, more preferably will have at least 92% sequence identity, more preferably will have at least 93% sequence identity, more preferably will have at least 94% sequence identity, more preferably will have at least 95% sequence identity, more preferably will have at least 96% sequence identity, more preferably will have at least 97% sequence identity, more preferably will have at least 98% sequence identity, more preferably will have at least 99% sequence identity with an HCV RNA polymerase polypeptide comprising an amino acid sequence comprising SEQ ID NO:1 or 2.

The term “active site,” as used herein, refers to a region of a molecule or molecular complex that, as a result of its shape, distribution of electrostatic charge, presentation of hydrogen-bond acceptors or hydrogen-bond donors, and/or distribution of nonpolar regions, favorably associates with RNA template primer molecules. Thus, an active site may include or consist of features such as cavities, surfaces, or interfaces between domains. A structural active site can include “in contact” amino acid residues as determined from examination of a three-dimensional structure. “Contact” can be determined using Van der Waals radii of atoms or by proximity sufficient to exclude solvent, typically water, from the space between the ligand and the molecule or molecular complex. In some embodiments, an HCV RNA polymerase residue in contact with an RNA template molecule is a residue that has one atom within about 5 Å of nucleic acid residue of the RNA template molecule. Alternatively, “in contact” residue may be those that have a loss of solvent accessible surface area of at least about 10 Å and, more preferably at least about 50 Å to about 300 Å. Loss of solvent accessible surface can be determined by the method of Lee and Richards (J. Mol. Biol., 1971, Feb. 14; 55(3):379-400) and similar algorithms known to those skilled in the art.

Some of the “in contact” amino acid residues, if substituted with another amino acid type, may not cause any change in a biochemical assay, a cell-based assay, or an in vivo assay used to define a functional binding site but may contribute to the formation of a three dimensional structure. A functional binding site includes amino acid residues that are identified as active site residues based upon loss or gain of function, for example, an increase in RNA polymerase activity or a decrease in resistance to RNA polymerase inhibitors. In some embodiments, the amino acid residues of a functional binding site are a subset of the amino acid residues of the structural binding site.

The term “HCV RNA polymerase active site” refers to a region of HCV RNA polymerase that can favorably associate with an RNA Template primer molecule. The term active site as used herein includes the region that performs the catalysis and formation of the phosphate bond that links the product to the incorporating nucleotide as well as residues that stabilize the incoming RNA template strand and the outgoing RNA dimer strand. The active site of HCV NS5B lies at the center of the protein and is formed by elements provided by all three subdomains of the enzyme, the “fingers,” the “thumb” and the “palm”. Some of the residues of the active site are described herein. The active site necessarily morphs such that elements important to de novo initiation provided by the “thumb” subdomain β-hairpin loop (Tyr448) move away from the active site, which permits elongation (growing the RNA product strand) to begin. Two metal ions Mg⁺² or Mn⁺² catalyze the formation of the phosphate backbone and they are stabilized in the active site by Asp318, Asp319, Asp220, and Thr221. Arg158 plays a role in the catalysis by stabilizing the incipient diphosphate leaving group. Asp225 appears to interact with the ribose 2′OH and 3′OH probably directing the nucleotide into the proper binding pocket for addition to the RNA daughter strand. Additionally, Ile160 appears to be the only residue to interact directly with the base (purine or pyrimidine) moiety of the nucleotide that is incoming along with that of the template strand nucleotide to which it is being paired by the polymerase. Finally, Ser282, which can mutate to a threonine upon development of resistance to the 2′Me nucleotide class, appears to play a structural role in stabilizing the fingerloop which presents both Arg158 and Ile160 into the active site.

A “structurally equivalent active site” is defined by a root mean square deviation from the structure coordinates of the backbone atoms of the amino acids that make up an active site of HCV RNA polymerase of at most about 0.70 Å, preferably about 0.5 Å.

“Crystal” as used herein, refers to one form of a solid state of matter in which atoms are arranged in a pattern that repeats periodically in three dimensions, typically forming a lattice. “Complementary or complement” as used herein, means the fit or relationship between two molecules that permits interaction, including for example, space, charge, three-dimensional configuration, and the like.

The term “corresponding” or “corresponds” refers to an amino acid residue or amino acid sequence that is found at the same position or positions in a sequence when the amino acid position or sequences are aligned with a reference sequence. In some embodiments, the reference sequence is a fragment of the HCV RNA polymerase having a sequence of SEQ ID NO:1 or 2. It will be appreciated that when the amino acid position or sequence is aligned with the reference sequence the numbering of the amino acids may differ from that of the reference sequence.

“Heavy atom derivative” as used herein, means a derivative produced by chemically modifying a crystal with a heavy atom such as Hg, Au, Se, Se methionines, or a halogen.

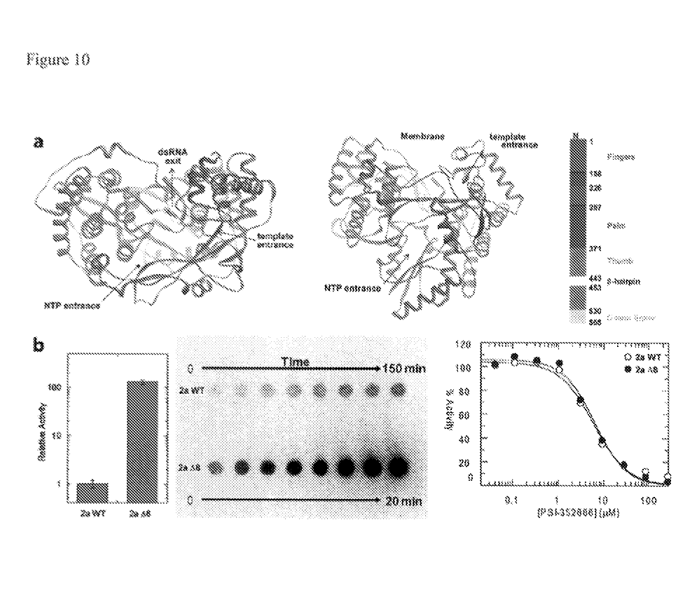

“Structural homolog” of HCV RNA polymerase used herein refers to a protein that contains one or more amino acid substitutions, deletions, additions, or rearrangements with respect to the amino acid sequence of, but that, when folded into its native conformation, exhibits or is reasonably expected to exhibit at least a portion of the tertiary (three-dimensional) structure of the HCV RNA polymerase. In some embodiments, a portion of the three-dimensional structure refers to structural domains of the HCV RNA polymerase including the “right hand” shape common to many polymerases along with easily recognized “fingers,” “palm” and “thumb” domains⁸⁻¹⁰ (FIG. 10a). For example, structurally homologous molecules can have substitutions, deletions or additions of one or more contiguous or noncontiguous amino acids, such as a loop or a domain. Structurally homologous molecules also include “modified” HCV RNA polymerase molecules that have been chemically or enzymatically derivatized at one or more constituent amino acid, including side-chain modifications, backbone modifications, and N- and C-terminal modifications including acetylation, hydroxylation, methylation, amidation, and the attachment of carbohydrate or lipid moieties, cofactors, and like modifications.

“RNA template primer molecules” as used herein, refers to a single ribonucleotide or a polynucleotide that associates with an active site on an HCV RNA polymerase. In some embodiments an RNA template primer molecule includes both a template strand and a primer strand. Symmetrical RNA template molecules include 4, 6, and 8 base pair ribonucleic acids such as those shown in Table 5. For example, in RNA 54, the RNA template primer molecule is partially double stranded including both a primer stand and a template strand; 5′-UACCGd-3′ template strand and 3′-dGCCAU-5′ primer strand. Fused template hairpin RNA constructs having about 10-25 ribonucleotides. Fused primer template hairpins include 3, 5, 6, 7, or 9 contiguous cytosines and guanosines separated by at least 4 ribonucleotides that allow formation of a hairpin structure. Specific embodiments of fused primer template hairpins are shown in Table 5. Both symmetric and nonsymmetric RNA template primer molecules are described herein.

“Molecular complex,” as used herein, refers to a combination of HCV RNA polymerase in a complex with RNA template primer molecules. In some embodiments, a molecular complex includes HCV RNA polymerase, RNA template, and RNA product strands.

“Machine-readable data storage medium,” as used herein, means a data storage material encoded with machine-readable data, wherein a machine is programmed with instructions for using such data and is capable of displaying data in the desired format, for example, a graphical three-dimensional representation of molecules or molecular complexes.

“Scalable,” as used herein, means the increasing or decreasing of distances between coordinates (configuration of points) by a scalar factor while keeping the angles essentially the same.

“Space group symmetry,” as used herein, means the whole symmetry of the crystal that combines the translational symmetry of a crystalline lattice with the point group symmetry. A space group is designated by a capital letter identifying the lattice type (P, A, F, etc.) followed by the point group symbol in which the rotation and reflection elements are extended to include screw axes and glide planes. Note that the point group symmetry for a given space group can be determined by removing the cell centering symbol of the space group and replacing all screw axes by similar rotation axes and replacing all glide planes with mirror planes. The point group symmetry for a space group describes the true symmetry of its reciprocal lattice.

“Unit cell,” as used herein, means the atoms in a crystal that are arranged in a regular repeating pattern, in which the smallest repeating unit is called the unit cell. The entire structure can be reconstructed from knowledge of the unit cell, which is characterized by three lengths (a, b and c) and three angles (α, β and γ). The quantities a and b are the lengths of the sides of the base of the cell and γ is the angle between these two sides. The quantity c is the height of the unit cell. The angles α and β describe the angles between the base and the vertical sides of the unit cell.

“X-ray diffraction pattern” means the pattern obtained from X-ray scattering of the periodic assembly of molecules or atoms in a crystal. X-ray crystallography is a technique that exploits the fact that X-rays are diffracted by crystals. X-rays have the proper wavelength (in the Ångstrom (Å) range, approximately 10⁻⁸ cm) to be scattered by the electron cloud of an atom of comparable size. Based on the diffraction pattern obtained from X-ray scattering of the periodic assembly of molecules or atoms in the crystal, the electron density can be reconstructed. Additional phase information can be extracted either from the diffraction data or from supplementing diffraction experiments to complete the reconstruction (the phase problem in crystallography). A model is then progressively built into the experimental electron density, refined against the data to produce an accurate molecular structure.

X-ray structure coordinates define a unique configuration of points in space. Those of skill in the art understand that a set of structure coordinates for a protein or a protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a configuration of points can be defined by increasing or decreasing the distances between coordinates by a scalar factor, while keeping the angles essentially the same.

“Crystal structure” generally refers to the three-dimensional or lattice spacing arrangement of repeating atomic or molecular units in a crystalline material. The crystal structure of a crystalline material can be determined by X-ray crystallographic methods, see for example, “Principles of Protein X-Ray Crystallography,” by Jan Drenth, Springer Advanced Texts in Chemistry, Springer Verlag; 2nd ed., February 1999, ISBN: 0387985875, and “Introduction to Macromolecular Crystallography,” by Alexander McPherson, Wiley-Liss, Oct. 18, 2002, ISBN: 0471251224.

### MODES FOR CARRYING OUT THE INVENTION

The present disclosure thus includes a crystalline form and a crystal structure of HCV RNA polymerase and HCV RNA polymerase in a complex with RNA template primer molecules. In some embodiments, a molecular complex includes HCV RNA polymerase, RNA template, and RNA product strands. In other aspects, the disclosure provides methods of using the crystal structures and structural coordinates to identify homologous proteins and to design or identify agents that can modulate the function of the HCV RNA polymerase and HCV RNA polymerase in a complex with RNA template primer molecules.

The present disclosure also includes the three-dimensional configuration of points derived from the structure coordinates of at least a portion of HCV RNA polymerase and HCV RNA polymerase in a complex with RNA template primer molecules, as well as structurally equivalent configurations, as described herein. The three-dimensional configuration includes points derived from structure coordinates representing the locations of a plurality of the amino acids defining the HCV RNA polymerase active site when it is not bound to substrate or when it is bound to a substrate. Likewise, the disclosure also includes the scalable three-dimensional configuration of points derived from structure coordinates of molecules or molecular complexes that are structurally homologous to HCV RNA polymerase and HCV RNA polymerase in a complex with RNA template primer molecules as well as structurally equivalent configurations. Structurally homologous molecules or molecular complexes are defined below.

Advantageously, structurally homologous molecules can be identified using the structure coordinates of the HCV RNA polymerase and HCV RNA polymerase in a complex with RNA template primer molecules according to a method of the disclosure. The configurations of points in space derived from structure coordinates according to the disclosure can be visualized as, for example, a holographic image, a stereodiagram, a model, or a computer-displayed image, and the disclosure thus includes such images, diagrams or models.

The crystal structure and structural coordinates can be used in methods, for example, for obtaining structural information of a related molecule, and for identifying and designing agents that modulate HCV RNA polymerase activity.

The coordinates of HCV RNA polymerase are provided in Table 1. The coordinates of HCV RNA polymerase in a complex with an RNA template primer strands are provided in Table 2 and Table 3.

### 1. HCV RNA Polymerase Polypeptides, Variants, and Nucleic Acids Encoding the Polymerase Polypeptides

### HCV RNA Polymerase Polypeptides

The present disclosure provides a description of HCV RNA polymerase polypeptides. HCV RNA polymerase refers, to any native (whether naturally occurring or synthetic) hepatitis C virus (HCV) polypeptide that is capable of binding to RNA molecules and synthesize RNA. The non-structural 5B (NS5B) protein, a 66 kDa protein of ˜590 amino acids found at the C-terminus of the virally encoded HCV polyprotein, provides the requisite RNA-dependent RNA polymerase (RdRp) functionality.⁶ The polymerase produces positive RNA strands for encapsidation into viral particles by using an intermediate negative RNA strand which it synthesizes from the initial positive strand RNA template provided by the virus. GTP-dependent de novo initiation is the preferred mode of nucleotide polymerization in vivo. Wild-type HCV RNA polymerase generally refers to a polypeptide having an amino acid sequence found in a naturally occurring HCV RNA polymerase and includes naturally occurring truncated or secreted forms, and variant forms. An example of a wild-type HCV RNA polymerase is a polypeptide comprising an amino acid sequence of SEQ ID NO:1 or 2.

HCV RNA polymerases from different genotypes of HCV genotypes are known. The alignment in Table 8 shows amino acid positions that are conserved and those that vary. The amino acid positions that vary can be changed without an expectation that functional activity will be changed. Other HCV RNA polymerase sequences can be used in homology modeling as described herein in order to identify RNA template molecules that may bind to or inhibit all of the HCV RNA polymerases.

The activity of HCV RNA polymerase can be determined by those of skill in the art and include methods to determine RNA synthesis activity in the presence or absence of inhibitors.

### HCV RNA Polymerase Variants

In one aspect of the disclosure, the disclosure describes HCV RNA polymerase variants. In some embodiments, the disclosure provides an isolated variant HCV RNA polymerase having at least 95% sequence identity to a polypeptide having the amino acid sequence of SEQ ID NO:1, wherein the polymerase variant has increased RNA synthesis activity as compared to HCV genotype 2a having an amino acid sequence of SEQ ID NO:1 or 3. In other embodiments, the HCV genotype 2a RNA polymerase variant has a sequence of SEQ ID NO:8. In some embodiments, the disclosure provides an isolated variant HCV RNA polymerase having at least 95% sequence identity to a polypeptide having the amino acid sequence of SEQ ID NO:2, wherein the polymerase variant has increased RNA synthesis activity as compared to HCV genotype 1b having an amino acid sequence of SEQ ID NO:2 or 4. In other embodiments, the HCV genotype 1b RNA polymerase variant has a sequence of SEQ ID NO:5, 6, or 7.

An HCV RNA polymerase variant refers to polypeptide that has a different sequence than a reference polypeptide. In some embodiments, the reference polypeptide is an HCV RNA polymerase comprising SEQ ID NO:1 or 2. Variants include “non-naturally” occurring variants. The variants include those polypeptides that have substitutions, additions or deletions.

In some embodiments, the HCV RNA polymerase has a deletion of 0 hairpin residues that correspond to amino acids 442-454 of HCV RNA polymerase genotype 2a of SEQ ID NO:3 or of HCV RNA polymerase genotype 1b of SEQ ID NO:4.

In other embodiments, a variant HCV RNA polymerase lacks a membrane binding region, for example, a deletion of a C-terminal peptide of at least 20 to 30 amino acids.

In some embodiments, a variant has one or more amino acid substitutions such as those that enhance solubility of the molecule. In some embodiments, at least one amino acid substitution is selected from the group consisting of an amino acid position corresponding to position 47, 86, 87, 114 of the HCV RNA polymerase having the amino acid sequence of SEQ ID NO:1, 2, 3 or 4, and mixtures thereof.

In some embodiments, a variant HCV RNA polymerase has an amino acid substitution at an amino acid position associated with resistance to inhibitors of RNA polymerase activity. In some embodiments, a variant HCV RNA polymerase has an amino acid substitution at an amino acid position 282. In some embodiments, a variant HCV RNA polymerase has an amino acid substitution at an amino acid position selected from the group consisting of 15, 223, 321 and mixtures thereof.

In some embodiments, a variant HCV RNA polymerase has an amino acid substitution in one or more of the active site residues. In some embodiments, a variant HCV RNA polymerase has an amino acid substitution at an amino acid position selected from the group consisting of Tyr448, Asp318, Asp319, Asp220, Thr221, Arg158, Asp225, Ile160, and Ser282 and mixtures thereof.

In some embodiments, the variants have increased RNA polymerase activity as compared the biological activity of wild-type HCV RNA polymerase. A number of variant HCV RNA polymerases are shown in Table 4.

Ordinarily, an HCV RNA polymerase variant polypeptide will have at least 80% sequence identity, more preferably will have at least 81% sequence identity, more preferably will have at least 82% sequence identity, more preferably will have at least 83% sequence identity, more preferably will have at least 84% sequence identity; more preferably will have at least 85% sequence identity, more preferably will have at least 86% sequence identity, more preferably will have at least 87% sequence identity, more preferably will have at least 88% sequence identity, more preferably will have at least 89% sequence identity, more preferably will have at least 90% sequence identity, more preferably will have at least 91% sequence identity, more preferably will have at least 92% sequence identity, more preferably will have at least 93% sequence identity, more preferably will have at least 94% sequence identity, more preferably will have at least 95% sequence identity, more preferably will have at least 96% sequence identity, more preferably will have at least 97% sequence identity, more preferably will have at least 98% sequence identity, more preferably will have at least 99% sequence identity with an HCV RNA polymerase polypeptide comprising an amino acid sequence comprising SEQ ID NO:1 or 2.

Variants can be prepared by synthetic or recombinant means. Methods for introducing changes such as amino acid substitutions, deletions, and insertions are known to those of skill in the art.

### HCV RNA Polymerase Nucleic Acids

In one aspect, the disclosure describes nucleic acids coding for HCV RNA polymerase polypeptides and variants. In some embodiments, the disclosure provides an isolated nucleic acid coding for an HCV genotype 2a RNA polymerase variant having at least 95% amino acid sequence identity to a polypeptide having the amino acid sequence of SEQ ID NO:1, wherein the polymerase variant has increased RNA synthesis activity as compared to HCV genotype 2a having an amino acid sequence of SEQ ID NO:1 or 3. In other embodiments, the isolated HCV genotype 2a RNA polymerase variant has a sequence of SEQ ID NO:8.

In another aspect, the disclosure describes nucleic acids coding for HCV RNA polymerase polypeptides and variants. In some embodiments, the disclosure provides an isolated nucleic acid coding for an HCV genotype 1b RNA polymerase variant having at least 95% amino acid sequence identity to a polypeptide having the amino acid sequence of SEQ ID NO:2, wherein the polymerase variant has increased RNA synthesis activity as compared to HCV genotype 1b having an amino acid sequence of SEQ ID NO:2 or 4. In other embodiments, the isolated HCV genotype 2a RNA polymerase variant has a sequence of SEQ ID NO:5, 6, or 7.

Nucleic acid sequences that code for HCV RNA polymerases form a variety of HCV genotypes and are known and readily available in databases such as GenBank. Vectors and methods for preparing such polypeptides are described herein. Methods of preparing variants of the HCV RNA polymerases can be conducted using standard methods such as site specific mutagenesis, cassette mutagenesis, PCR based mutagenesis, and the like.

### Fusion Proteins

HCV RNA polymerase polypeptides, variants, or structural homolog or portions thereof, may be fused to a heterologous polypeptide or compound. The heterologous polypeptide is a polypeptide that has a different function than that of the HCV RNA polymerase. Examples of heterologous polypeptide include polypeptides that may act as carriers, may extend half life, may act as epitope tags, and may provide ways to detect or purify the fusion protein. Heterologous polypeptides include KLH, albumin, salvage receptor binding epitopes, immunoglobulin constant regions, and peptide tags. Peptide tags useful for detection or purification include FLAG, gD protein, polyhistidine tags, hemagglutinin from influenza virus, T7 tag, S tag, Strep tag, chloramphenicol acetyl transferase, biotin, glutathione-S transferase, green fluorescent protein, and maltose binding protein. Compounds that can be combined with HCV RNA polymerase, variants or structural homolog or portions thereof, include radioactive labels, protecting groups, and carbohydrate or lipid moieties.

### Polynucleotides, Vectors and Host Cells

HCV RNA polymerase variants or fragments thereof can be prepared by introducing appropriate nucleotide changes into DNA encoding HCV RNA polymerase, or by synthesis of the desired polypeptide variants.

Polynucleotide sequences encoding the polypeptides described herein can be obtained using standard recombinant techniques. Desired polynucleotide sequences may be isolated and sequenced from appropriate source cells. Alternatively, polynucleotides can be synthesized using nucleotide synthesizer or PCR techniques. Once obtained, sequences encoding the polypeptides or variant polypeptides are inserted into a recombinant vector capable of replicating and expressing heterologous polynucleotides in a host cell. Many vectors that are available and known in the art can be used for the purpose of the present invention. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication (in particular when the vector is inserted into a prokaryotic cell), a selection marker gene, a promoter, a ribosome binding site (RBS), a signal sequence, the heterologous nucleic acid insert and a transcription termination sequence.

In general, plasmid vectors containing replicon and control sequences, which are derived from a species compatible with the host cell, are used in connection with these hosts. The vector ordinarily carries a replication site, as well as marking sequences, which are capable of providing phenotypic selection in transformed cells. For example, a useful vector is a pET-28a-based vector. In addition, phage vectors containing replicon and control sequences that are compatible with the host microorganism can be used as transforming vectors in connection with these hosts.

Either constitutive or inducible promoters can be used in the present invention, in accordance with the needs of a particular situation, which can be ascertained by one skilled in the art. A large number of promoters recognized by a variety of potential host cells are well known.

Eukaryotic host cell systems are also well established in the art. Examples of invertebrate cells include insect cells such as Drosophila S2 and Spodoptera Sf9, as well as plants and plant cells. Examples of useful mammalian host cell lines include Chinese hamster ovary (CHO) and COS cells. More specific examples include monkey kidney CV1 line transformed by SV40 (COS-7, ATCC CRL 1651); Chinese hamster ovary cells/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); and mouse mammary tumor (MMT 060562, ATCC CCL51).

### Polypeptide Production

Host cells are transformed or transfected with the above-described expression vectors and cultured in conventional nutrient media modified as appropriate for inducing promoters, selecting transformants, or amplifying the genes encoding the desired sequences.

Transfection refers to the taking up of an expression vector by a host cell whether or not any coding sequences are in fact expressed. Numerous methods of transfection are known to the ordinarily skilled artisan, for example, CaPO₄ precipitation and electroporation. Successful transfection is generally recognized when any indication of the operation of this vector occurs within the host cell.

Eukaryotic cells used to produce the polypeptides of the invention are grown in media known in the art and suitable for culture of the selected host cells. Optionally the culture medium may contain one or more reducing agents selected from the group consisting of glutathione, cysteine, cystamine, thioglycollate, dithioerythritol and dithiothreitol. If an inducible promoter is used in the expression vector, protein expression is induced under conditions suitable for the activation of the promoter. A variety of inducers may be used, according to the vector construct employed, as is known in the art.

Eukaryotic host cells are cultured under conditions suitable for expression of the HCV RNA polymerase polypeptides. The host cells used to produce the polypeptides may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in one or more of Ham et al., 1979, Meth. Enz. 58:44, Barnes et al., 1980, Anal. Biochem. 102: 255, U.S. Pat. No. 4,767,704, U.S. Pat. No. 4,657,866, U.S. Pat. No. 4,927,762, U.S. Pat. No. 4,560,655, or U.S. Pat. No. 5,122,469, WO 90/103430, WO 87/00195, and U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES™), nucleotides (such as adenosine and thymidine), antibiotics (such as GENTAMYCIN™), trace elements (defined as inorganic compounds usually present at final concentrations in the micromolar range), and glucose or an equivalent energy source. Other supplements may also be included at appropriate concentrations that would be known to those skilled in the art. The culture conditions, such as temperature, pH, and the like, are those previously used with the host cell selected for expression, and will be apparent to the ordinarily skilled artisan.

Polypeptides described herein expressed in a host cell may be secreted into and/or recovered from the periplasm of the host cells. Protein recovery typically involves disrupting the microorganism, generally by such means as osmotic shock, sonication, or lysis. Once cells are disrupted, cell debris or whole cells may be removed by centrifugation or filtration. The proteins may be further purified, for example, by affinity resin chromatography. Alternatively, proteins can be transported into the culture media and isolated therefrom. Cells may be removed from the culture and the culture supernatant being filtered and concentrated for further purification of the proteins produced. The expressed polypeptides can be further isolated and identified using commonly known methods such as fractionation on immunoaffinity or ion-exchange columns; ethanol precipitation; reverse phase HPLC; chromatography on silica or on a cation exchange resin such as DEAE; chromatofocusing; SDS-PAGE; ammonium sulfate precipitation; gel filtration using, for example, Sephadex G-75; hydrophobic affinity resins, ligand affinity using a suitable antigen immobilized on a matrix and Western blot assay.

Polypeptides that are produced may be purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In some embodiments, the polypeptide is purified by nickel affinity chromatography. The cells are lysed in 20 mM Tris, pH 8.0, 0.5 M NaCl, 2 mMTCEP, 5 mM Mg(OAc)₂, 20% glycerol with protease inhibitors, lysozyme, and Benzonase with stirring on ice followed by sonication. After clarification, the soluble protein fraction is loaded onto a Ni-NTA FF column equilibrated in 20 mM Tris, pH 8.0, 0.5 M NaCl, 2 mMTCEP, 20% glycerol. The protein is eluted using a gradient protocol and elution buffer supplemented with 0.5 M imidazole. Pooled fractions are collected and dialyzed into 10 mM HEPES (pH 7.5), 0.4 M NaCl, and 2 mM TCEP. The protein is concentrated with a 50 kDa MWCO Amicon Ultra filter to remove lower molecular contaminants.

### 2. Crystals and Crystal Structures

In another aspect, the present disclosure provides a crystalline form of and a crystal structure of the HCV RNA polymerase and HCV RNA polymerase in a complex with RNA template primer molecules. In some embodiments, the disclosure provides a crystal comprising an HCV RNA polymerase comprising a variant HCV RNA polymerase having at least 95% sequence identity to a polypeptide having the amino acid sequence of SEQ ID NO:8, wherein the polymerase variant has increased RNA synthesis activity as compared to HCV genotype 2a having an amino acid sequence of SEQ ID NO:1 or 3. In other embodiments, the disclosure provides a crystal comprising an HCV RNA polymerase RNA template primer complex comprising a variant HCV RNA polymerase having at least 95% sequence identity to a polypeptide having the amino acid sequence of SEQ ID NO:8, wherein the polymerase variant has increased RNA synthesis activity as compared to HCV genotype 2a having an amino acid sequence of SEQ ID NO:1 or 3 and RNA template primer molecules. In some embodiments, the HCV RNA polymerase has an amino acid sequence of SEQ ID NO:8. Crystals can be combined with a carrier to form a composition. Crystals of HCV RNA polymerase may also be a useful way to store, or concentrate HCV RNA polymerase.

Another aspect of the disclosure provides methods of crystallizing HCV RNA polymerase and HCV RNA polymerase in a complex with RNA template primer molecules. In some embodiments, a method of crystallizing an HCV RNA polymerase comprises isolating an HCV RNA polymerase, variant or fragment thereof, and contacting the HCV RNA polymerase with about 30% pentaerythritol ethoxylate, 50 mM BisTris pH 6.5, 50 mM ammonium sulfate until crystals form. In a specific embodiment, the HCV RNA polymerase comprises a variant HCV RNA polymerase having at least 95% sequence identity to a polypeptide having the amino acid sequence of SEQ ID NO:8, wherein the polymerase variant has increased RNA synthesis activity as compared to HCV genotype 2a having an amino acid sequence of SEQ ID NO:1 or 3.

In other embodiments, a method of crystallizing an HCV RNA polymerase comprises a) isolating an HCV RNA polymerase or fragment or variant thereof; b) contacting the HCV RNA polymerase with about 0.2 M ammonium acetate, 0.1 M Bis Tris pH 5.5, 25% PEG 3350 until crystals form; and c) contacting the crystal with RNA template primer molecules. In a specific embodiment, the HCV RNA polymerase comprises a variant HCV RNA polymerase having at least 95% sequence identity to a polypeptide having the amino acid sequence of SEQ ID NO:8, wherein the polymerase variant has increased RNA synthesis activity as compared to HCV genotype 2a having an amino acid sequence of SEQ ID NO:1 or 3.

In some embodiments, the RNA template molecule comprises a 4, 6, 8 RNA primer template molecule or RNA primer hairpin template. Symmetrical RNA primer template molecules include 4, 6, and 8 base pair ribonucleic acid, such as those shown in Table 5. Fused primer template hairpin RNA constructs having about 10-25 ribonucleotides. Fused primer template hairpins include 3, 5, 6, 7, or 9 contiguous cytosines and guanosines separated by at least 4 ribonucleotides that allow formation of a hairpin structure. Specific embodiments of fused primer template hairpins are shown in Table 5.

In other embodiments, the disclosure provides a crystalline HCV RNA polymerase comprising a variant HCV RNA polymerase having the amino acid sequence of SEQ ID NO:8 having a space group symmetry of P6₅ and comprising a unit cell having the dimensions of a=b and is about 140.69 Å, c is about 92.63 Å, α=β and is about 90°, and γ is about 120°. In some embodiments, the disclosure provides a crystalline HCV RNA polymerase RNA template primer complex comprising a variant HCV RNA polymerase having the amino acid sequence of SEQ ID NO:8 having a space group symmetry of P6₅ and comprising a unit cell having the dimensions of a=b is about 143.27.69 Å, c is about 92.19 Å or 91.5 Å, α=β and is about 90°, and γ is about 120°.

The three dimensional coordinates of a crystal of HCV RNA polymerase comprising a variant HCV RNA polymerase having the amino acid sequence of SEQ ID NO:8 are provided in Table 1. The three dimensional coordinates of a crystal of HCV RNA polymerase RNA template comprising a variant HCV RNA polymerase having the amino acid sequence of SEQ ID NO:8 and RNA template primer molecules is provided in Table 2 and Table 3. The term “structure coordinates” refers to Cartesian coordinates derived from mathematical equations related to the patterns obtained on diffraction of a monochromatic beam of X-rays by the atoms (scattering centers) of an HCV RNA polymerase and HCV RNA polymerase in a complex with RNA template primer molecules in crystal form. The diffraction data are used to calculate an electron density map of the repeating unit of the crystal. The electron density maps are then used to establish the positions of the individual atoms of the HCV RNA polymerase and HCV RNA polymerase in a complex with RNA template primer molecules.

Slight variations in structure coordinates can be generated by mathematically manipulating the HCV RNA polymerase and HCV RNA polymerase in a complex with RNA template primer molecules complex structure coordinates. For example, the structure coordinates as set forth in Tables 1-3 could be manipulated by crystallographic permutations of the structure coordinates, fractionalization of the structure coordinates, integer additions or subtractions to sets of the structure coordinates, inversion of the structure coordinates, or any combination of the above. Alternatively, modifications in the crystal structure due to mutations, additions, substitutions, deletions, and combinations thereof, of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail herein.

It should be noted that slight variations in individual structure coordinates of the HCV RNA polymerase and HCV RNA polymerase in a complex with RNA template primer molecules would not be expected to significantly alter the nature of chemical entities such as ligands that could associate with a binding site or other structural features of HCV RNA polymerase. In this context, the phrase “associating with” refers to a condition of proximity between a ligand, or portions thereof, and an HCV RNA polymerase or portions thereof. The association may be non-covalent, wherein the juxtaposition is energetically favored by hydrogen bonding, van der Waals forces, and/or electrostatic interactions, or it may be covalent.

### HCV RNA Polymerase Structure and Active Site

The JFH1 isolate of genotype 2a is the only cloned HCV strain capable of efficient replication in cell culture as well as in vivo²³. A construct of 2a JFH1 NS5B with the β-hairpin loop replaced with a Gly-Gly linker (“2a Δ8”) was observed to be >100-fold more active than wild-type 2a in de novo RNA synthesis assays, capable of binding RNA in thermofluor analysis and resulted in a similar IC₅₀ value for chain termination with PSI-352666 (6 μM) relative to wild-type 2a. A 2.5 Å resolution apo crystal structure of 2a Δ8 was obtained which revealed substantial structural changes relative to previously determined 2a NS5B structures²³⁻²⁵ with an overall r.m.s.d value of 1.8 Å.

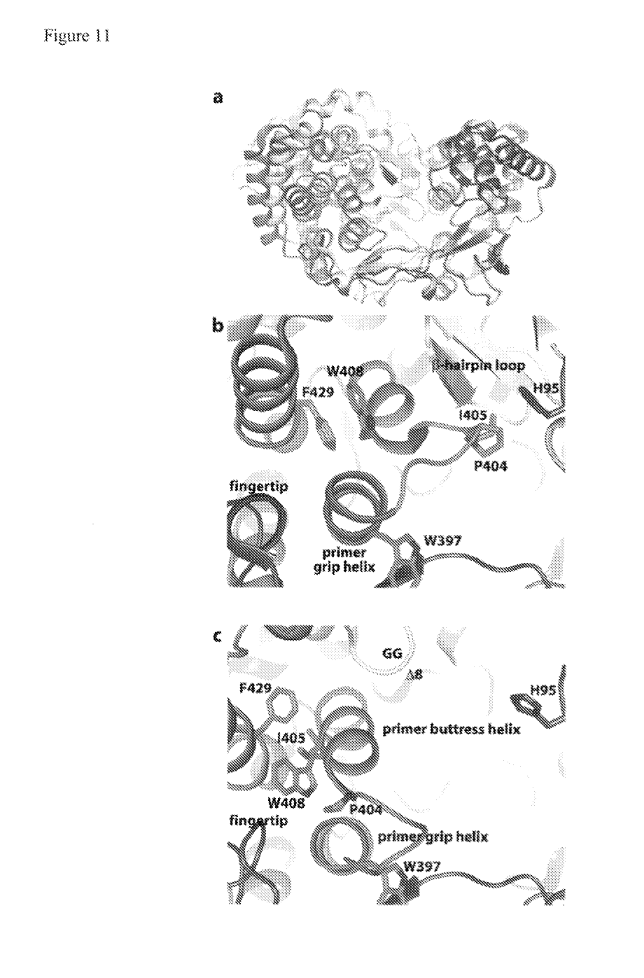

The 2a Δ8 Gly444-Gly445 linker was ordered and Phe551 was the last ordered residue. Alignment of the palm and fingers domains of a closed apo 2a structure²³ with the apo 2a Δ8 structure shows an overall ˜20° movement of the thumb domain. The lack of the β-hairpin loop, the disorder of the C-terminal linker region, and the movement of the thumb domain combine to generate a large cavity in the center of the polymerase. The thumb domain movement is accompanied by significant reordering of residues 397-412 which connect the primer grip helix with the primer buttress helix (FIG. 11). In particular, Ile405, which was previously detailed to be important for de novo initiation across all genotypes²³, moved more than 12 Å away from the β-hairpin loop in the closed wild-type structure to extend the primer buttress helix and pack on top of the highly conserved Trp408 (FIG. 11). Trp408 stacked on top of the nearly invariant Phe429 in the closed wild-type structure, and both residues adopt different rotamer conformations in the 2a Δ8 structure. In addition, the highly conserved Pro404, which contacts His95 of the finger domain in the closed apo structure (FIG. 11b), forms a key turn in the loop while packing on top of the main chain of Trp397 in the apo 2a Δ8 structure (FIG. 11c). This loop reordering may be critical to the transition from de novo initiation with GTP to elongation of the growing primer-template RNA. Comparison of the apo 2a Δ8 structure with other RdRp ternary complexes¹⁷⁻¹⁹ suggested that in this open conformation, HCV NS5B may be able to bind primer-template RNA.

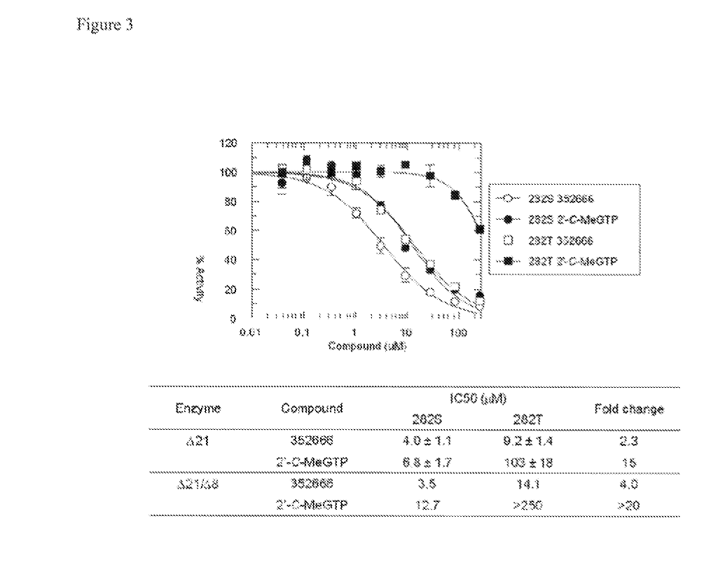

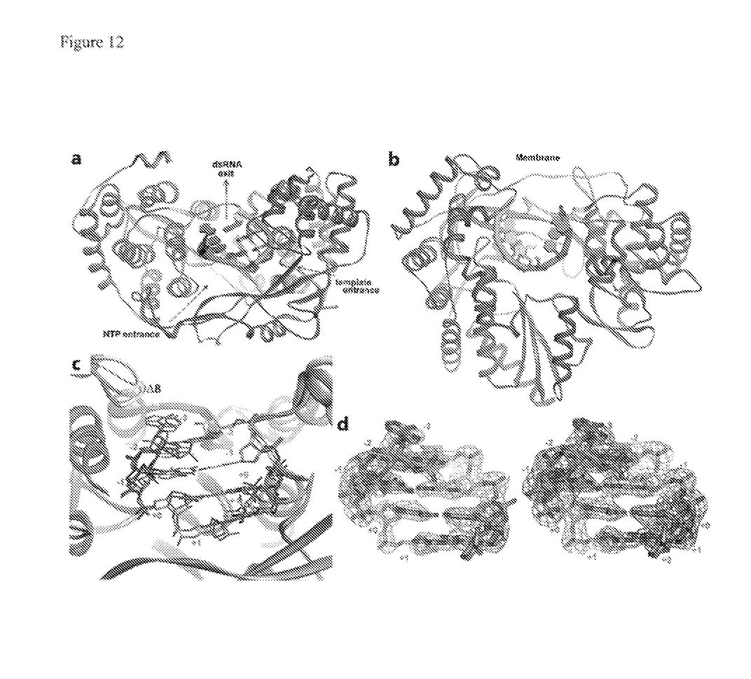

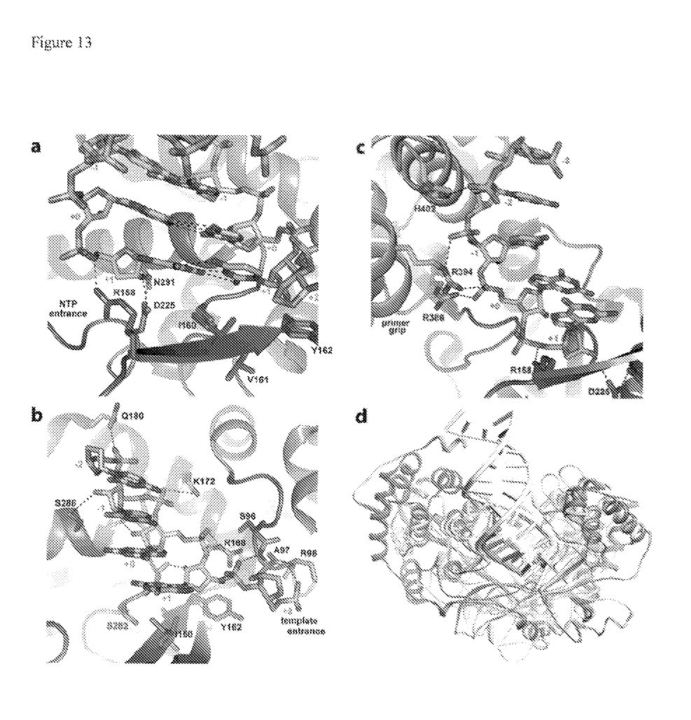

Crystals of apo 2a Δ8 were soaked with symmetrical primer-template RNA pairs and structures were determined at 2.9 Å and 3.0 Å resolution (FIG. 12). A-form RNA was readily apparent in the resulting electron density maps (FIG. 12d), clearly showing the differences in purine/pyrimidine pairings of the two RNA sequences. Both symmetrical primer-template RNA pairs were designed as obligate chain terminators with either a 3′-dG or a 2′,3′-ddC, and thus, unsurprisingly, a product state, pretranslocation registry was observed in both complexes (FIG. 3c). None of the nucleobase hydrogen bond acceptors or donors is recognized by the polymerase, indicating sequence independent recognition by the polymerase. The nucleobase of the pairing nucleotide of the template strand (residue+1 by convention) stacks on top of the strictly conserved Ile160, as predicted⁹, while the sugar stacks on top of Tyr162, which is conserved as Tyr or Phe (FIG. 13a). When the pairing nucleotide is a pyrimidine, residue+1 of the primer strand (equivalent to the incoming NTP) also packs with Ile160 (FIG. 13a), possibly accounting for some of the differences in purine/pyrimidine analog triphosphate inhibitor activity. All of the phosphates and 2′-hydroxyls of the template strand are recognized by NS5B (FIG. 13b), demonstrating the importance of an RNA template for HCV. Likewise, the phosphates of the primer strand are recognized by Arg158 of the finger domain and the primer grip helix of the thumb domain, while the primer buttress helix forms van der Waals interactions with several primer strand sugars. The 2′-hydroxyl of primer residue+1 of the product, pretranslocation state, which resides at the same position as the incoming NTP in the substrate registry, is recognized by the side chain of Asp225 (FIG. 13c). As both structures contain a 3′-deoxy terminal residue, the other carboxylate oxygen of Asp225 is free to hydrogen bond with Asn291. The equivalent residue (Asp238) of the poliovirus RdRp was shown to adopt different conformations depending on the incoming NTP, translocation state, and presence of divalent metal ions¹⁸. None of the other 2′-hydroxyls of the primer strand are recognized by NS5B, consistent with reduced activity with DNA primers²⁶.

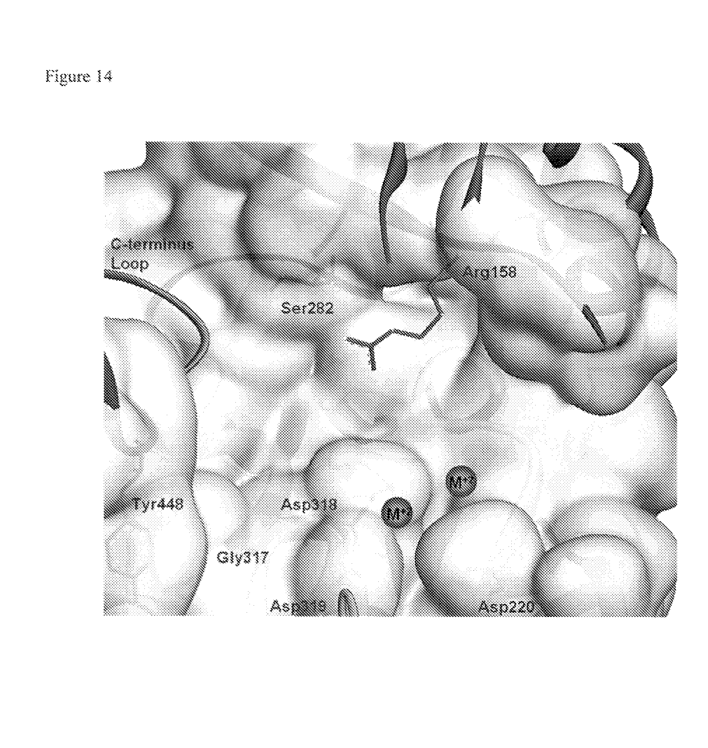

The active site of HCV NS5B lies at the center of the protein and is formed by elements provided by all three subdomains of the enzyme, the “fingers,” the “thumb” and the “palm”. The active site necessarily morphs such that elements important to de novo initiation provided by the “thumb” subdomain β-hairpin loop (Tyr448) move away from the active site which permits elongation (growing the RNA product strand) to begin (the structure described in the Nature article excised the β-hairpin loop including the Tyr448). Two metal ions Mg⁺² or Mn⁺² catalyze the formation of the phosphate backbone and they are stabilized in the active site by Asp318, Asp319, Asp220, and Thr221. Arg158 plays a role in the catalysis by stabilizing the incipient diphosphate leaving group. Based on the structure, Asp225 appears to interact with the ribose 2′OH and 3′OH probably directing the nucleotide into the proper binding pocket for addition to the RNA daughter strand. Additionally, Ile160 appears to be the only residue to interact directly with the base (purine or pyrimidine) moiety of the nucleotide that is incoming along with that of the template strand nucleotide to which it is being paired by the polymerase. Finally, Ser282, which can mutate to a threonine upon development of resistance to the 2′Me nucleotide class, appears to play a structural role in stabilizing the fingerloop which presents both Arg158 and Ile160 into the active site (see, e.g., FIG. 14).

Another aspect of the disclosure provides a molecule or molecular complex comprising at least a portion of an unbound HCV RNA polymerase active site of a polypeptide having an amino acid sequence of SEQ ID NO:8, wherein the active site comprises at least one amino acid residue corresponding to an amino acid residue in a position of HCV RNA polymerase selected from the group consisting of Tyr448, Asp318, Asp319, Asp220, Thr221, Arg158, Asp225, Ile160, Ser282, and mixtures thereof, and the at least one amino acid residue is defined by a set of points having a root mean square deviation of less than about 0.70 Å from points representing the backbone atoms of the amino acids as represented by the structure coordinates listed in Table 1, 2, and/or 3.

Another aspect of the disclosure provides a three-dimensional configuration of points wherein at least a portion of the points are derived from structure coordinates of Table 1, 2, and/or 3 representing locations of the backbone atoms of amino acids defining the HCV RNA polymerase active site. In some embodiments, the disclosure provides a three-dimensional configuration of points displayed as a holographic image, a stereodiagram, a model, or a computer-displayed image, at least a portion of the points derived from structure coordinates listed in Table 1, 2, and/or 3, comprising an HCV RNA polymerase active site, wherein the HCV RNA polymerase forms a crystal having the space group symmetry P6₅.

### 3. Structurally Equivalent Crystal Structures

Various computational analyses can be used to determine whether a molecule or portions of the molecule defining structure features are “structurally equivalent,” defined in terms of its three-dimensional structure, to all or part of HCV RNA polymerase or HCV RNA polymerase bound to RNA template molecule. Such analyses may be carried out in current software applications, such as the Molecular Similarity application of QUANTA (Molecular Simulations Inc., San Diego, Calif.), Version 4.1, and as described in the accompanying User's Guide.

The Molecular Similarity application permits comparisons between different structures, different conformations of the same structure, and different parts of the same structure. A procedure used in Molecular Similarity to compare structures comprises: 1) loading the structures to be compared; 2) defining the atom equivalences in these structures; 3) performing a fitting operation; and 4) analyzing the results.

One structure is identified as the target (i.e., the fixed structure); all remaining structures are working structures (i.e., moving structures). As atom equivalency within QUANTA is defined by user input, for the purpose of this disclosure equivalent atoms are defined as protein backbone atoms (N, Cα, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in Angstroms, is reported by QUANTA.

Structurally equivalent crystal structures have portions of the two molecules that are substantially identical, within an acceptable margin of error. The margin of error can be calculated by methods known to those of skill in the art; in some embodiments, any molecule or molecular complex or any portion thereof, that has a root mean square deviation of conserved residue backbone atoms (N, Cα, C, O) of less than about 0.70 Å, preferably 0.5 Å. For example, structurally equivalent molecules or molecular complexes are those that are defined by the entire set of structure coordinates listed in Tables 1, 2, and/or 3±a root mean square deviation from the conserved backbone atoms of those amino acids of not more than 0.70 Å, preferably 0.5 Å. The term “root mean square deviation” means the square root of the arithmetic mean of the squares of the deviations. It is a way to express the deviation or variation from a trend or object. For purposes of this disclosure, the “root mean square deviation” defines the variation in the backbone of a protein from the backbone of HCV RNA polymerase or HCV RNA polymerase in a complex with RNA template primer molecules (as defined by the structure coordinates of the complex as described herein) or a defining structural feature thereof.

### 4. Structurally Homologous Molecules, Molecular Complexes, and Crystal Structures

Structure coordinates can be used to aid in obtaining structural information about another crystallized molecule or molecular complex. The method of the disclosure allows determination of at least a portion of the three-dimensional structure of molecules or molecular complexes that contain one or more structural features that are similar to structural features of at least a portion of the HCV RNA polymerase or HCV RNA polymerase in a complex with RNA template primer molecules. These molecules are referred to herein as “structurally homologous” to HCV RNA polymerase or HCV RNA polymerase in a complex with RNA template primer molecules. Similar structural features can include, for example, regions of amino acid identity, conserved active site or binding site motifs, and similarly arranged secondary structural elements.

Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Two amino acid sequences are compared using the BLAST program, version 2.0.9, of the BLAST 2 search algorithm, as described by Tatusova et al. (56), and available at www.ncbi.nlm.nih.gov/BLAST/. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.” In some embodiments, a structurally homologous molecule is a protein that has an amino acid sequence having at least 80% identity with a wild type or recombinant amino acid sequence of HCV RNA polymerase having a sequence of SEQ ID NO:1 or 2. More preferably, a protein that is structurally homologous to HCV RNA polymerase includes at least one contiguous stretch of at least 50 amino acids that has at least 80% amino acid sequence identity with the analogous portion of the wild type or recombinant HCV RNA polymerase. Methods for generating structural information about the structurally homologous molecule or molecular complex are well known and include, for example, molecular replacement techniques. An alignment of HCV RNA polymerases from other viral genotypes is provided in Table 8.

Therefore, in another embodiment this disclosure provides a method of utilizing molecular replacement to obtain structural information about a molecule or molecular complex whose structure is unknown comprising: (a) generating an X-ray diffraction pattern from a crystallized molecule or molecular complex of unknown or incompletely known structure; and (b) applying at least a portion of the structural coordinates of HCV RNA polymerase or HCV RNA polymerase in a complex with RNA template primer molecules to the X-ray diffraction pattern to generate a three-dimensional electron density map of the molecule or molecular complex whose structure is unknown or incompletely known.

By using molecular replacement, all or part of the structure coordinates of HCV RNA polymerase or HCV RNA polymerase in a complex with RNA template primer molecules as provided by this disclosure can be used to determine the unsolved structure of a crystallized molecule or molecular complex more quickly and efficiently than attempting to determine such information ab initio.

Molecular replacement can provide an accurate estimation of the phases for an unknown or incompletely known structure. Phases are one factor in equations that are used to solve crystal structures, and this factor cannot be determined directly. Obtaining accurate values for the phases, by methods other than molecular replacement, can be a time-consuming process that involves iterative cycles of approximations and refinements and greatly hinders the solution of crystal structures. However, when the crystal structure of a protein containing at least a structurally homologous portion has been solved, molecular replacement using the known structure provide a useful estimate of the phases for the unknown or incompletely known structure.

Thus, this method involves generating a preliminary model of a molecule or molecular complex whose structure coordinates are unknown, by orienting and positioning the relevant portion of the HCV RNA polymerase or HCV RNA polymerase in a complex with RNA template primer molecules within the unit cell of the crystal of the unknown molecule or molecular complex. This orientation or positioning is conducted so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure. This map, in turn, can be subjected to established and well-known model building and structure refinement techniques to provide a final, accurate structure of the unknown crystallized molecule or molecular complex (see, for example, Lattman, 1985 Methods in Enzymology 115:55-77).

Structural information about a portion of any crystallized molecule or molecular complex that is sufficiently structurally homologous to a portion of HCV RNA polymerase or HCV RNA polymerase in a complex with RNA template primer molecules can be solved by this method.

A heavy atom derivative of HCV RNA polymerase or HCV RNA polymerase in a complex with RNA template primer molecules is also included as a homolog. The term “heavy atom derivative” refers to derivatives of HCV RNA polymerase or HCV RNA polymerase in a complex with RNA template primer molecules produced by chemically modifying a crystal of HCV RNA polymerase or HCV RNA polymerase in a complex with RNA template primer molecules. In practice, a crystal is soaked in a solution containing heavy metal atom salts, or organometallic compounds, e.g., lead chloride, gold thiomalate, selenium methionine, thiomersal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the protein (Blundell, et al., 1976, Protein Crystallography, Academic Press, San Diego, Calif.).

The structure coordinates of HCV RNA polymerase or HCV RNA polymerase in a complex with RNA template primer molecules are also particularly useful to solve or model the structure of crystals of HCV RNA polymerase variants or homologs which are co-complexed with a variety of RNA template primer molecules. This approach enables the determination of the optimal sites for interaction between candidate HCV RNA polymerase inhibitors including inhibitors of resistant HCV RNS polymerases. This information provides an additional tool for determining more efficient binding interactions, for example, increased hydrophobic or polar interactions, between HCV RNA polymerase or HCV RNA polymerase in a complex with RNA template primer molecules. For example, high-resolution X-ray diffraction data collected from crystals exposed to different types of solvent allows the determination of where each type of solvent molecule resides. Small molecules that bind tightly to those sites can then be designed and synthesized and tested for their HCV RNA polymerase affinity, and/or inhibition activity.

All of the complexes referred to above may be studied using well-known X-ray diffraction techniques and may be refined versus 1.5-3.5 Å resolution X-ray data to an R-factor of about 0.30 or less using computer software, such as X-PLOR (Yale University, distributed by Molecular Simulations, Inc.) (see, for example, Blundell et al. 1976. Protein Crystallography, Academic Press, San Diego, Calif., and Methods in Enzymology, Vol. 114 and 115, H. W. Wyckoff et al., eds., Academic Press (1985)). This information may thus be used to optimize known HCV RNA polymerase inhibitors and more importantly, to design new modulators.

The disclosure also includes the unique three-dimensional configuration defined by a set of points defined by the structure coordinates for a molecule or molecular complex structurally homologous to HCV RNA polymerase or HCV RNA polymerase in a complex with RNA template primer molecules as determined using the method of the present disclosure, structurally equivalent configurations, and magnetic storage media including such set of structure coordinates.

5. Methods for Identification of Modulators of HCV RNA Polymerase or HCV RNA Polymerase in a Complex with RNA Template Primer Molecules

In another aspect, a candidate modulator can be identified using a biological assay such as binding to HCV RNA polymerase or HCV RNA polymerase in a complex with RNA template primer molecules. The candidate modulator can then serve as a model to design similar agents and/or to modify the candidate modulator, for example, to improve characteristics such as binding to HCV RNA polymerase. Design or modification of candidate modulators can be accomplished using the crystal structure coordinates and available software.

In some embodiments, the disclosure provides a method of assessing agents that are antagonists or agonists of HCV RNA polymerase active site comprising applying at least a portion of the crystallography coordinates of Table 1, 2, or 3 to a computer algorithm that generates a three-dimensional model of HCV RNA polymerase active site suitable for designing molecules that are antagonists, and searching a molecular structure database to identify potential antagonists of HCV RNA polymerase active site. In other embodiments, a method further comprises synthesizing or obtaining the antagonist, contacting the antagonist with HCV RNA polymerase, and selecting the antagonist that modulates the activity of HCV RNA polymerase.

In some embodiments, computer implemented methods are also provided. An embodiment of such a method involves a computer-assisted method for identifying an agent that modulates HCV RNA polymerase active activity comprising modifying at least one nucleic acid residue in RNA template primer molecules, and performing a fitting operation between the modified RNA template molecules and the structural coordinates of at least one amino acid residue of an HCV RNA polymerase active site as set forth in Table 1, 2, or 3. Other embodiments involve a computer-assisted method for identifying an agent that modulates HCV RNA polymerase activity comprising (a) providing a computer modeling application with a set of structure coordinates of Table 1, 2 or 3 defining at least a portion of an HCV RNA polymerase active site; (b) providing the computer modeling application with a set of structure coordinates for a test agent; and (c) modeling the structure of (a) complexed with (b) to determine if the test agent associates with the HCV RNA polymerase active site.

Yet other embodiments involve a computer-assisted method for designing an agent that binds the HCV RNA polymerase, comprising: (a) providing a computer modeling application with a set of structural coordinates of Table 1, 2, or 3 defining at least a portion of the HCV RNA polymerase active site; and (b) modeling the structural coordinates of (a) to identify an agent that contacts at least one amino acid residue in the HCV RNA polymerase active site.

### Binding Site and Other Structural Features

The present disclosure provides information inter alia about the shape and structure of the active site of HCV RNA polymerase in the presence or absence of a substrate. The association of natural ligands or substrates with the active sites of their corresponding receptors or enzymes is the basis of many biological mechanisms of action. Similarly, many drugs exert their biological effects through association with the binding sites of receptors and enzymes. Such associations may occur with all or any part of the binding site. An understanding of such associations helps lead to the design of drugs having more favorable associations with their target, and thus improved biological effects. Therefore, this information is valuable in designing potential modulators of HCV RNA polymerase active sites, as discussed in more detail below.

In some embodiments, the active site of HCV RNA polymerase for a template molecule comprises, consists essentially of, or consists of at least one amino acid residue corresponding to an amino acid residue in a position of HCV RNA polymerase at amino acid Tyr448, Asp318, Asp319, Asp220, Thr221, Arg158, Asp225, Ile160, Ser282, and mixtures thereof. The active site of HCV RNA polymerase may be defined by those amino acids whose backbone atoms are situated within about 5 Å of one or more constituent atoms of a bound substrate or ligand or that lose solvent accessible surface area to due to a bound substrate or ligand. In some embodiments, the amino acid residues on HCV RNA polymerase that lose at least 10 to about 300 Å, more preferably about 50 Å to 300 Å, of solvent accessible surface area are amino acid residues that form a part or all of the HCV RNA polymerase active site. Preferably, the active site in unbound conformation of HCV RNA polymerase comprises all of these amino acid residues.

### Rational Drug Design

Computational techniques can be used to screen, identify, select, design ligands, and combinations thereof, capable of associating with HCV RNA polymerase or structurally homologous molecules. Candidate modulators of HCV RNA polymerase may be identified using functional assays, such as binding to HCV RNA polymerase, and novel modulators designed based on the structure of the candidate molecules so identified. Knowledge of the structure coordinates for HCV RNA polymerase permits, for example, the design, the identification of synthetic compounds, and like processes, and the design, the identification of other molecules and like processes that have a shape complementary to the conformation of the HCV RNA polymerase active sites. In particular, computational techniques can be used to identify or design ligands, such as agonists and/or antagonists that associate with an HCV RNA polymerase active site. Antagonists may bind to or interfere with all or a portion of an active site of HCV RNA polymerase, and can be competitive, noncompetitive, or uncompetitive inhibitors. Once identified and screened for biological activity, these agonists, antagonists, and combinations thereof, may be used therapeutically and/or prophylactically, for example, to block HCV RNA polymerase activity and thus prevent the onset and/or further progression of diseases associated with HCV RNA polymerase activity. Structure-activity data for analogues of ligands that bind to or interfere with HCV RNA polymerase can also be obtained computationally.

In other embodiments, a criterion that may be utilized in the design of modulators is whether the modulator can fit into the active site cavity on HCV RNA polymerase. The volume of the cavity can be determined by placing atoms in the entrance of the pocket close to the surface and using a program like GRASP to calculate the volume of those atoms. Another criterion is whether the antagonist strengthens interactions with the amino acid residues in the active site such as Tyr448, Asp318, Asp319, Asp220, Thr221, Arg158, Asp225, Ile160, and Ser282. In some embodiments, an inhibitor will be designed to interact with an amino acid at least one or all residues in the active site.

Data stored in a machine-readable storage medium that is capable of displaying a graphical three-dimensional representation of the structure of HCV RNA polymerase or a structurally homologous molecule or molecular complex, as identified herein, or portions thereof may thus be advantageously used for drug discovery. The structure coordinates of the ligand are used to generate a three-dimensional image that can be computationally fit to the three-dimensional image of HCV RNA polymerase or a complex of HCV RNA polymerase and an RNA template molecule, or a structurally homologous molecule. The three-dimensional molecular structure encoded by the data in the data storage medium can then be computationally evaluated for its ability to associate with ligands. When the molecular structures encoded by the data are displayed in a graphical three-dimensional representation on a computer screen, the protein structure can also be visually inspected for potential association with ligands.

One embodiment of the method of drug design involves evaluating the potential association of a candidate ligand with HCV RNA polymerase or a structurally homologous molecule or homologous complex, particularly with at least one amino acid residue in an active site or a portion of the binding site. The method of drug design thus includes computationally evaluating the potential of a selected ligand to associate with any of the molecules or molecular complexes set forth above. This method includes the steps of: (a) employing computational means, for example, such as a programmable computer including the appropriate software known in the art or as disclosed herein, to perform a fitting operation between the selected ligand and a ligand binding site or a subside of the ligand binding site of the molecule or molecular complex, and (b) analyzing the results of the fitting operation to quantify the association between the ligand and the ligand binding site. Optionally, the method further comprises analyzing the ability of the selected ligand to interact with amino acids in the HCV RNA polymerase active site and/or subside. The method may also further comprise optimizing the fit of the ligand for the binding site of HCV RNA polymerase genotype 2a as compared to other HCV RNA polymerases. Optionally, the selected ligand can be synthesized, crystallized with HCV RNA polymerase, and further modifications to selected ligand can be made to enhance inhibitory activity or fit in the active site.

In another embodiment, the method of drug design involves computer-assisted design of ligand that associates with HCV RNA polymerase, its homologs, or portions thereof. Ligands can be designed in a step-wise fashion, one fragment at a time, or may be designed as a whole or de novo. Ligands can be designed based on the structure of molecules that can modulate at least one biological function of HCV RNA polymerase such as PSI 3526666 or 2′ C-MeGTP. In addition, the inhibitors can be modeled on other known inhibitors of HCV RNA polymerase.

Optionally, the potential binding of a ligand to an HCV RNA polymerase active site is analyzed using computer modeling techniques prior to the actual synthesis and testing of the ligand. If these computational experiments suggest insufficient interaction and association between it and the HCV RNA polymerase active site, testing of the ligand is obviated. However, if computer modeling indicates a strong interaction, the molecule may then be synthesized and tested for its ability to bind to or interfere with an HCV RNA polymerase active site. Binding assays to determine if a compound actually modulates HCV RNA polymerase activity can also be performed and are well known in the art.

Docking may be accomplished using software such as QUANTA and SYBYL, followed by energy minimization and molecular dynamics with standard molecular mechanics force fields, such as CHARMM and AMBER. Specialized computer programs may also assist in the process of selecting ligands. Examples include GRID (Hubbard, S. 1999. Nature Strict. Biol. 6:711-14); MCSS (Miranker and Karplus (1991) Proteins 11:29-34) available from Molecular Simulations, San Diego, Calif.; AUTODOCK (Goodsell et al. 1990. Proteins 8:195-202) available from Scripps Research Institute, La Jolla, Calif.; and DOCK (Kuntz et al. 1982 J. Mol. Biol. 161:269-88) available from University of California, San Francisco, Calif.

Once a compound has been designed or selected by the above methods, the efficiency with which that ligand may bind to or interfere with an HCV RNA polymerase active site may be tested and optimized by computational evaluation. A ligand designed or selected as binding to or interfering with a binding site may be further computationally optimized so that in its bound state it would preferably lack repulsive electrostatic interaction with the target enzyme and with the surrounding water molecules. Such noncomplementary electrostatic interactions include repulsive charge-charge, dipole-dipole, and charge-dipole interactions. Specific computer software is available to evaluate compound deformation energy and electrostatic interactions. Examples of programs designed for such uses include: Gaussian 94, revision C (M. J. Frisch, Gaussian, Inc., Pittsburgh, Pa.); AMBER, version 4.1 (P. A. Kollman, University of California at San Francisco, Calif.); QUANTA/CHARMM (Molecular Simulations, Inc., San Diego, Calif.); Insight II/Discover (Molecular Simulations, Inc., San Diego, Calif.); DelPhi (Molecular Simulations, Inc., San Diego, Calif.); and AMSOL (Quantum Chemistry Program Exchange, Indiana University). These programs can be implemented, for instance, using a Silicon Graphics workstation, such as an Indigo2 with IMPACT graphics. Other hardware systems and software packages will be known to those skilled in the art.

Another approach encompassed by this disclosure is the computational screening of small molecule databases for ligands or compounds that can bind in whole, or in part, to an HCV RNA polymerase active site whether in bound or unbound conformation. In this screening, the quality of fit of such ligands to the binding site may be judged either by shape complementarity or by estimated interaction energy (Meng et al., 1992. J. Comp. Chem. 13:505-24).

Another method involves assessing agents that are antagonists or agonists of the HCV RNA polymerase. A method comprises applying at least a portion of the crystallography coordinates of Tables 1, 2, or 3 to a computer algorithm that generates a three-dimensional model of HCV RNA polymerase complexed with RNA template primer molecules suitable for designing molecules that are antagonists or agonists and searching a molecular structure database to identify potential antagonists or agonists. The method may further comprise synthesizing or obtaining the agonist or antagonist and contacting the agonist or antagonist with the HCV RNA polymerase and selecting the antagonist or agonist that modulates the HCV RNA polymerase activity compared to a control without the agonist or antagonists and/or selecting the antagonist or agonist that binds to the HCV RNA polymerase.

### 6. Machine-Readable Storage Media

Transformation of the structure coordinates for all or a portion of HCV RNA polymerase or HCV RNA polymerase in a complex with RNA template primer molecules, or one of its active sites, or structurally homologous molecules as defined below, or for the structural equivalents of any of these molecules or molecular complexes as defined above, into three-dimensional graphical representations of the molecule or complex can be conveniently achieved through the use of commercially-available software.

The disclosure thus further provides a machine-readable storage medium including a data storage material encoded with machine-readable data wherein a machine programmed with instructions for using said data displays a graphical three-dimensional representation of any of the molecule or molecular complexes of this disclosure that have been described above. In a preferred embodiment, the machine-readable data storage medium includes a data storage material encoded with machine-readable data wherein a machine programmed with instructions for using the abovementioned data displays a graphical three-dimensional representation of a molecule or molecular complex including all or any parts of an HCV RNA polymerase or HCV RNA polymerase in a complex with RNA template primer molecules. In another preferred embodiment, the machine-readable data storage medium includes a data storage material encoded with machine readable data wherein a machine programmed with instructions for using the data displays a graphical three-dimensional representation of a molecule or molecular complex±a root mean square deviation from the atoms of the amino acids of not more than 0.05 Å.

In an alternative embodiment, the machine-readable data storage medium includes a data storage material encoded with a first set of machine readable data which includes the Fourier transform of structure coordinates, and wherein a machine programmed with instructions for using the data is combined with a second set of machine readable data including the X-ray diffraction pattern of a molecule or molecular complex to determine at least a portion of the structure coordinates corresponding to the second set of machine readable data.

For example, a system for reading a data storage medium may include a computer including a central processing unit (“CPU”), a working memory which may be, for example, RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, track balls, touch pads, etc.), one or more input lines, and one or more output lines, all of which are interconnected by a conventional bidirectional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as mobile devices, consumer electronics and appliances.

Input hardware may be coupled to the computer by input lines and may be implemented in a variety of ways. Machine-readable data of this disclosure may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of a binding site of this disclosure using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this disclosure. Such programs are discussed in reference to the computational methods of drug discovery as described herein. References to components of the hardware system are included as appropriate throughout the following description of the data storage medium.

Machine-readable storage devices useful in the present disclosure include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

### EXAMPLES

### Example 1

### Protein Expression and Purification

For our studies, constructs of wild-type HCV polymerase genotype 1b BK isolate and HCV polymerase genotype 2a JFH-1 isolate were designed as shown in Table 4.

TABLE 4

| Protein | | | SEQ ID |
|---|---|---|---|
| Construct | Structure | Amino Acid Sequence | NO: |
| 1b WT | 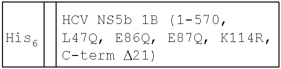 | MASHHHHHHSMSYTWTGALITPCAAEES KLPINALSNSLLRHHNMVYATTSRSAGQ RQKKVTFDRLQVLDDHYRDVLKEMKAKA STVKAKLLSVQQACKLTPPHSAKSKFGY GAKDVRNLSSRAVNHIHSVWKDLLEDTV TPIDTTIMAKNEVFCVQPEKGGRKPARL IVFPDLGVRVCEKMALYDVVSTLPQVVM GSSYGFQYSPGQRVEFLVNTWKSKKNPM GFSYDTRCFDSTVTENDIRVEESIYQCC DLAPEARQAIKSLTERLYIGGPLTNSKG QNCGYRRCRASGVLTTSCGNTLTCYLKA SAACRAAKLQDCTMLVNGDDLVVICESA GTQEDAASLRVFTEAMTRYSAPPGDPPQ PEYDLELITSCSSNVSVAHDASGKRVYY LTRDPTTPLARAAWETARHTPVNSWLGN IIMYAPTLWARMILMTHFFSILLAQEQL EKALDCQIYGACYSIEPLDLPQIIERLH GLSAFSLHSYSPGEINRVASCLRKLGVP PLRVWRHRARSVRARLLSQGGRAATCGK YLFNWAVKTKLKLTPIPAASQLDLSGWF VAGYSGGDIYHSLSRARPR | 4 |
| 1b S282T | 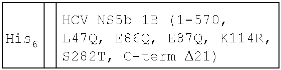 | MASHHHHHHSMSYTWTGALITPCAAEES KLPINALSNSLLRHHNMVYATTSRSAGQ RQKKVTFDRLQVLDDHYRDVLKEMKAKA STVKAKLLSVQQACKLTPPHSAKSKFGY GAKDVRNLSSRAVNHIHSVWKDLLEDTV TPIDTTIMAKNEVFCVQPEKGGRKPARL IVFPDLGVRVCEKMALYDVVSTLPQVVM GSSYGFQYSPGQRVEFLVNTWKSKKNPM GFSYDTRCFDSTVTENDIRVEESIYQCC DLAPEARQAIKSLTERLYIGGPLTNSKG QNCGYRRCRA [symbol] GVLTTSCGNTLTCYLKA SAACRAAKLQDCTMLVNGDDLVVICESA GTQEDAASLRVFTEAMTRYSAPPGDPPQ PEYDLELITSCSSNVSVAHDASGKRVYY LTRDPTTPLARAAWETARHTPVNSWLGN IIMYAPTLWARMILMTHFFSILLAQEQL EKALDCQIYGACYSIEPLDLPQIIERLH GLSAFSLHSYSPGEINRVASCLRKLGVP PLRVWRHRARSVRARLLSQGGRAATCGK YLFNWAVKTKLKLTPIPAASQLDLSGWF VAGYSGGDIYHSLSRARPR | 5 |
| 1bΔ8 | 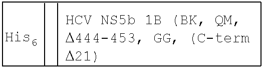 | 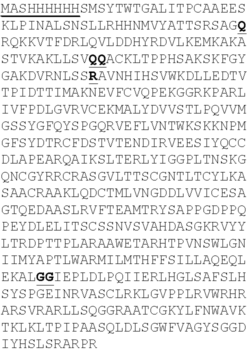 | 6 |
| 1bΔ8 S282T | 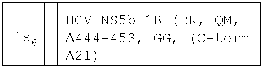 | 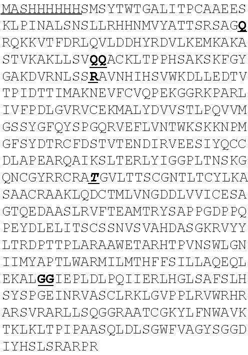 | 7 |
| 2a WT | 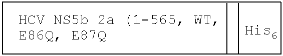 | 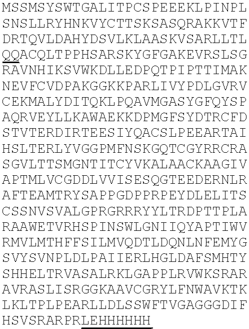 | 3 |
| 2aΔ8 | 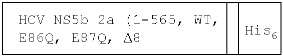 | 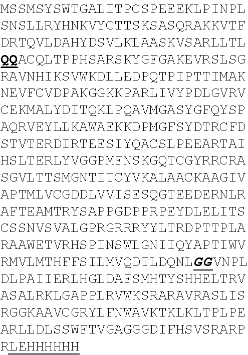 | 8 |

Nucleic acid constructs coding for wild-type HCV polymerase of HCV genotype 1b BK isolate were modified to code for a polymerase with the N-terminal 21 amino acids removed and replaced with a noncleavable hexahistidine tag. The nucleic acid constructs were cloned into a pET-28a-derived vector. Construct “1b WT” contained four surface solubilization mutations that resulted in amino acid substitutions L47Q, E86Q, E87Q, and K114R. Construct “1b S282T” was the same as 1b WT but coded for a polypeptide with a S282T resistance mutation. Construct “1bΔ8” used 1b WT as the template, but β-hairpin residues 444-453 were removed and replaced with a Gly-Gly linker. In Table 4, the residues replaced by the Gly-Gly linker are shaded in 1bWt. Construct “1bΔ8 S282T” used 1b Δ8 as the template, and further coded for a S282T resistance mutation.

Nucleic acid constructs of wild-type HCV polymerase of HCV genotype 2a JFH-1 isolate were modified to code for a polymerase with the C-terminal 21 amino acids removed and replaced with a noncleavable hexahistidine tag. The nucleic acid constructs were cloned into a pET-28a-derived vector. In construct “2a WT,” two surface solubilization mutations E86Q and E87Q were introduced via site-directed mutagenesis. In construct “2a Δ8”, the construct for 2aWT was used as a based template in which the β-hairpin was removed and replaced with a Gly-Gly linker. In Table 4, the residues replaced by the Gly-Gly linker are shaded in 2aWT. The constructs in which the β-hairpin was removed and replaced with a Gly-Gly linker were designed in GeneComposer and cloned by site-directed mutagenesis.

The clones were transformed into Rosetta DE3 E. coli cells (Novagen). Recombinant protein was expressed in the Overnight Expression Autoinduction System (VWR) at 22° C. overnight. Cells were harvested by centrifugation at 5000×g for 20 minutes and the cell pellet was resuspended in 20 mM Tris, pH 8.0, 500 mM NaCl, 20% glycerol, 2 mM TCEP, and 10 mM imidazole. The lysate was stirred in the presence of Benzonase and egg white lysozyme for 1 hour at 4° C. followed by clarification by centrifugation at 18,000 rpm for 40 minutes. The protein was purified by nickel immobilized affinity chromatography resulting in ˜90% pure protein as determined by Biorad Experion and SDS PAGE analysis. The protein was concentrated to 3.9-4.4 mg/mL (˜60 μM) in 20 mM Tris, pH 8.0, 500 mM NaCl, 20% glycerol, 2 mM TCEP and ˜200 mM imidazole.

### Example 2

### RNA Constructs

A series of symmetrical primer-template RNA constructs and fused primer-template hairpin RNA constructs were designed for use in HCV activity assays (Example 3), thermofluor analysis (Example 4), and crystallography (Example 5) of the HCV polymerase constructs from Example 1. The constructs were produced as previously reported (20, 21). Table 5 shows the RNA constructs produced. The shaded regions identify the base-pairing regions.

TABLE 5
Symmetrical primer-template RNA constructs

| | | SEQ |
|---|---|---|
| | | ID |
| 4 base-pair RNA | Identifier | NO: |
| 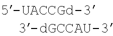 | RNA54, d = 3′-dG | 9 |
| 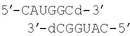 | RNA62, d = 2′, 3′- ddC | 10 |
| | | SEQ |
| | | ID |
| 6 base-pair RNA | Identifier | NO: |
| 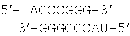 | RNA27 | 11 |
| 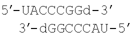 | RNA30, d = 3′-dG | 12 |
| | | SEQ |
| | | ID |
| 8 base-pair RNA | Identifier | NO: |
| 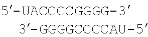 | RNA29 | 13 |
| 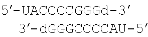 | RNA32, d = 3′-dG | 14 |

Fused primer-template hairpin RNA constructs

| | | SEQ |
|---|---|---|
| | | ID |
| Construct | Identifier | NO: |
| 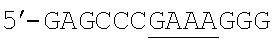 | RNA35 (13-mer, 3 bp) | 15 |
| 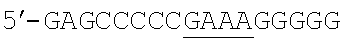 | RNA37 (17-mer, 5 bp) | 16 |
| 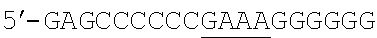 | RNA38 (19-mer, 6-bp) | 17 |
| 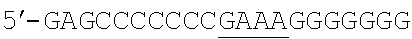 | RNA39 (21-mer, 7-bp) | 18 |
| 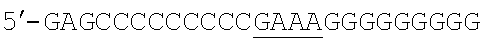 | RNA41 (25-mer, 9-bp) | 19 |

### Example 3

### HCV Activity Assays

Assays for de novo RNA synthesis activity and chain termination were performed on the HCV polymerase constructs from Example 1.

### Methods

HCV activity assays were performed in a 200 μL mixture containing 1 μM of the four natural ribonucleotides, [α-³²P]UTP, 20 ng/μL of genotype 1b (−) IRES RNA template, 1 unit/μL of SUPERase.In (Ambion, Austin, Tex.), 40 ng/μL of NS5B, 5 mM MgCl₂, and 2 mM DTT in 50 mM HEPES buffer (pH 7.5). The reaction was incubated at 27° C. and 20 μL aliquots were taken at the desired time points and quenched by mixing in 80 μL of stop solution (12.5 mM EDTA, 2.25 M NaCl, and 225 mM sodium citrate).

For constructs 2aWT and 2aΔ8, inhibition assays were performed in a 20 μL mixture containing varying concentrations of PSI-352666 or 2′-C-MeGTP, 5 μM of the four natural ribonucleotides, [α-³²P]UTP, 20 ng/μL of genotype 1b (−) IRES RNA template, 1 unit/μL of SUPERase.In (Ambion, Austin, Tex.), 40 ng/μL of NS5B, 5 mM MgCl₂, and 2 mM DTT in 50 mM HEPES buffer (pH 7.5).

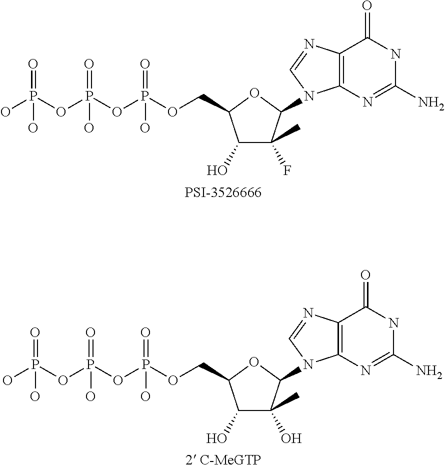 [SMILES: C[C@@]1(F)[C@H](O)[C@@H](COP(=O)(O)OP(=O)(O)OP(=O)(O)O)O[C@H]1n1cnc2c(=O)[nH]c(N)nc21 ; C[C@@]1(O)[C@H](O)[C@@H](COP(=O)(O)OP(=O)(O)OP(=O)(O)O)O[C@H]1n1cnc2c(=O)[nH]c(N)nc21]

The reaction was incubated at 27° C. and quenched by adding 80 μL of stop solution after 60 minutes incubation with 2aWT or 15 minutes incubation with 2aΔ8. The radioactive RNA products were separated from unreacted substrates using a Hybond N+ membrane (GE Healthcare) as described previously²⁸. The products were visualized and quantified using a phosphorimager. The reaction rates and the IC₅₀ values were calculated using GraphFit (Erithacus Software, Horley, Surrey, UK).

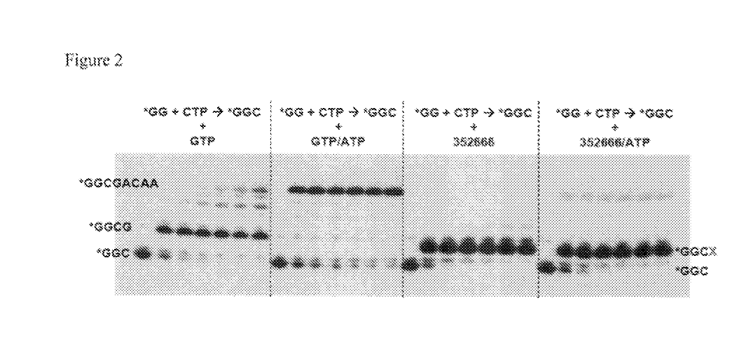

For constructs 1bWT and 1bΔ8, inhibition assays were performed in a 120 μL mixture containing 5 μM of the four natural ribonucleotides, [α-³²P]UTP, 20 ng/μL of genotype 1b (−) IRES RNA template, 1 unit/μL of SUPERase.In (Ambion, Austin, Tex.), 40 ng/μL of NS5B, 5 mM MgCl₂, and 2 mM DTT in 50 mM HEPES buffer (pH 7.5). The reaction was incubated at 27° C. and a 20 μL aliquot was taken at 0, 30, 60, 90, and 120 minute time points and quenched by mixing with 80 μL of stop solution (12.5 mM EDTA, 2.25 M NaCl, and 225 mM sodium citrate). The radioactive RNA products were separated from unreacted substrates using a Hybond N+ membrane (GE Healthcare) as described previously (FIG. 2). The products were visualized and quantified using a phosphorimager. The reaction rates and IC 50 values were calculated using GraphFit.

Thermofluor analysis of HCV polymerases 1bWT, 1bΔ8, 2aWT, and 2aΔ8 was performed as previously described (36, 37) in the absence and presence of symmetrical primer-template pairs or fused hairpin primer-template RNA as described in Example 2.

### Results

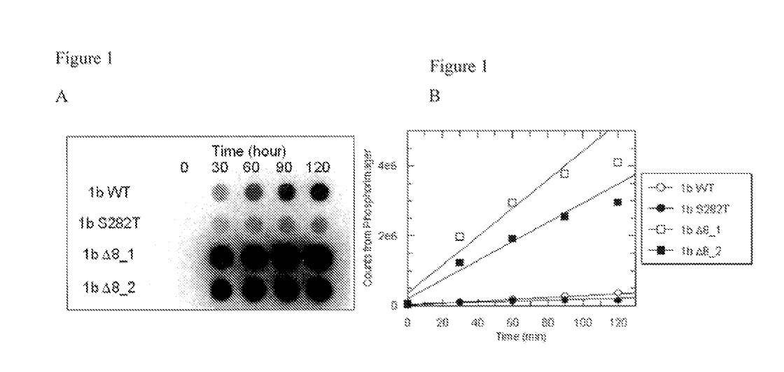

A greater than 10-fold increase in de novo RNA synthesis for 1bΔ8 samples with a Gly-Gly linker was observed on the Hybond membrane (FIG. 1A). Reaction rates were graphed as shown in FIG. 1B. The 1bΔ8 samples showed a significantly greater reaction rate at all time points as compared to 1b wild type and resistant polymerase. Table 6 shows a comparison of the relative activity of each of the enzymes, showing that the 1bΔ8 samples exhibited a 10-15 fold increase in relative polymerase activity.

TABLE 6

| Enzyme | Relative activity |
|---|---|
| 1b WT | 1 |
| 1b S282T | 0.51 |
| 1b Δ8_1 | 14.9 |
| 1b Δ8_2 | 10.1 |

This construct was also observed to be susceptible to chain-termination, and similar IC₅₀ values were obtained for the GTP analogues PSI-3526666 (4 μM) and 2′-C-MeGTP (7-13 μM) for both 1b wild-type (1b WT) and 1b Δ8. FIG. 2 shows chain termination of HCV NS5B polymerase 1b Δ8 construct in the presence of PSI-352666 over a 60 minute time period.

IC₅₀ determinations of chain termination reactions of HCV polymerase 1b WT, 1b S282T, 1b Δ8, and 1b Δ8 S282T with PSI-352666 or 2′-C-MeGTP in the presence of varying concentrations of PSI-3526666 or 2′-C-MeGTP are shown in FIG. 3. Upon introduction of the most commonly identified resistance mutation, S282T, similar inhibitory patterns for either the 1b WT or the 1b Δ8 construct were noted for these same compounds (PSI-352666, IC₅₀=9-14 μM, a ˜2.5-fold increase in IC₅₀; 2′-C-MeGTP IC₅₀=>100 μM; a >10-fold increase). The results show that constructs with the resistant mutation are less susceptible to chain termination inhibition by PSI-3526666. Constructs with the deletion and the resistant mutation show the most resistance to chain termination by 2′-C-MeGTP. These results suggest that constructs with the Δ8 mutation are good models of the WT in that they respond similarly to chain termination by the test inhibitors and have similar response to the S282 mutation.

Construct 2aΔ8 with the β-hairpin looped replaced with the Gly-Gly linker was observed to be >100-fold more active that wild-type 2a (2aWT) in de novo synthesis assays (FIG. 10b). Time-dependent formation of the radiolabeled products is shown in the blot (FIG. 10b). The IC₅₀ value for chain termination with PSI-3526666 (6 μM) of 2aΔ8 was found to be similar to 2aWT. At right in FIG. 10b, the activity for both 2a WT and 2a Δ8 were measured in the presence of the nucleotide triphosphate analog inhibitor PSI-3526666, which resulted in IC₅₀ value of 6.05±0.82 μM for 2a WT and 6.41±0.75 μM for 2aΔ8.

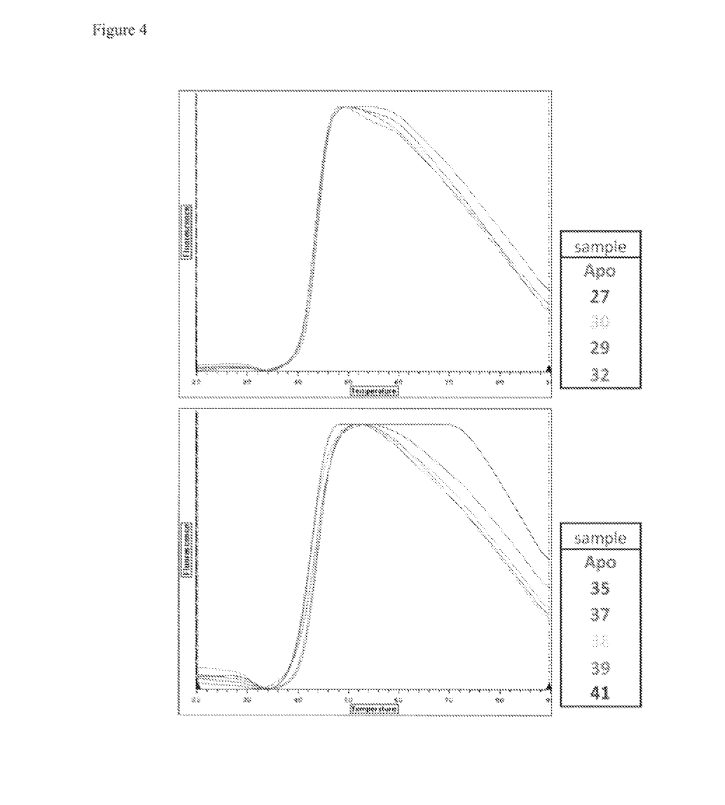

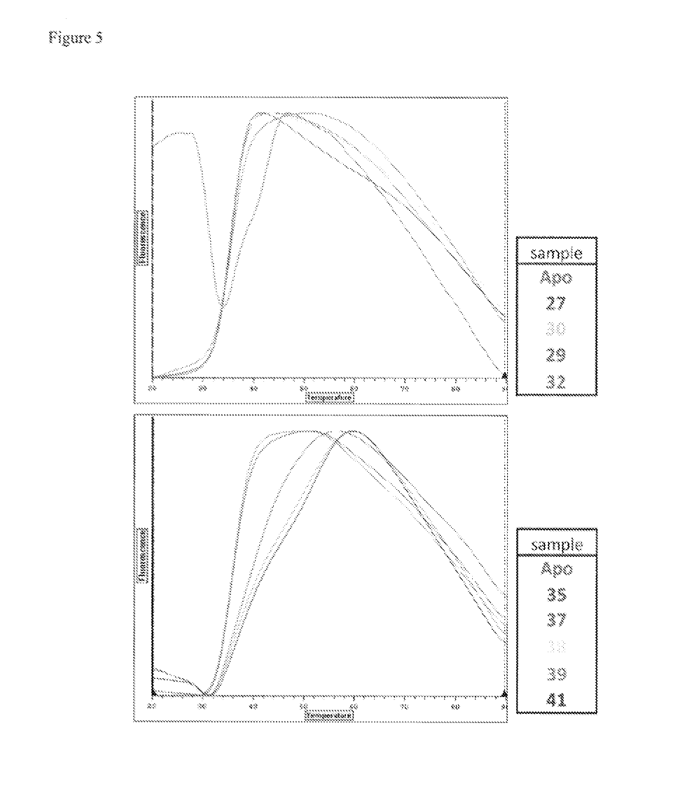

We performed thermofluor analysis^{36,37} on HCV polymerases 1b WT and 1b Δ8 in the absence and presence of symmetrical primer-template pairs or fused hairpin primer-template RNA. For 1b WT little or no change is observed in the melting temperature in the presence or absence of RNA (Tₘ˜44° C., FIG. 4). For 1b Δ8, we observed a destabilization of the polymerase (Tₘ˜35° C.) relative to 1b WT, but significant stabilization of up to +18° C. in the presence of either symmetrical primer-template pairs or fused hairpin primer-template RNA (FIG. 5).

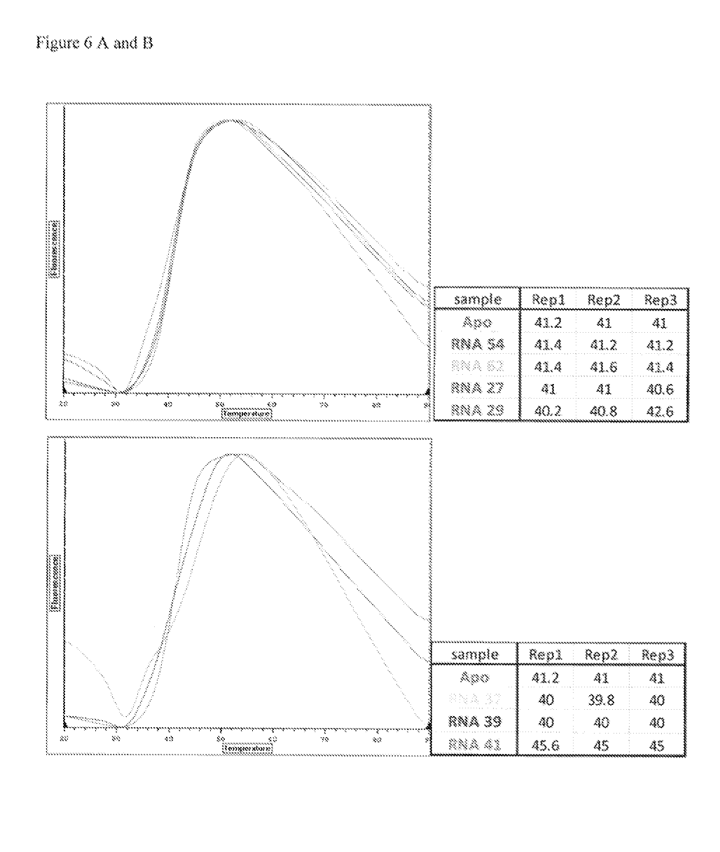

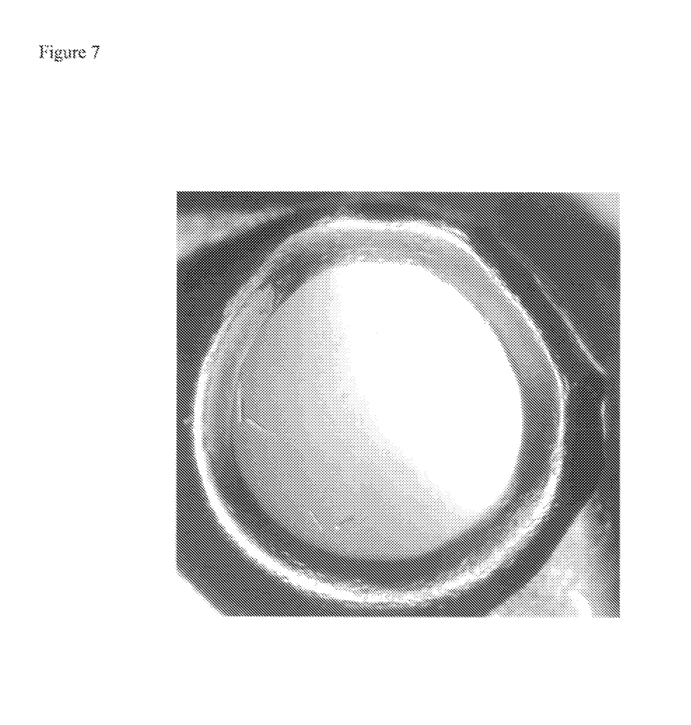

As was observed with genotype 1b HCV polymerase, we observed little or no change in the melting temperature for 2a WT in the presence or absence of RNA using thermofluor analysis (FIG. 6). For 2a Δ8, we again observed destabilization of the polymerase (Tₘ˜35° C.) relative to 2a WT (Tₘ˜41° C.). We observed significant stabilization in the presence of either symmetrical primer-template pairs or fused hairpin primer-template RNA (FIG. 7). The degree of stabilization correlates well with the number of base-pairs in the construct, demonstrating that larger RNAs form more interactions with themselves and with NS5B.

An HCV NS5B 1b BK construct in which residues 442-456 had been excised was synthesized. While this 1b Δ14 polymerase had reduced activity (data not shown), it provided the foundation for interest in a report that replacement of residues 444-453 (Δ8) in this β-hairpin loop with a Tyr-Gly linker in HCV NS5B genotype 1b (“1b Δ8”) resulted in a 17-fold increase in primer extension activity over wild-type and the ability to bind primer-template RNA^{20,21}. A >10-fold increase in de novo RNA synthesis for 1b Δ8 with a Gly-Gly linker (FIG. 1) as well as evidence for RNA binding via thermofluor analysis (FIGS. 4 and 5) was observed. This construct was susceptible to chain-termination, and similar IC₅₀ values were obtained for the GTP analogues PSI-352666 (4 μM) and 2′-C-MeGTP (7-13 μM) for both 1b wild-type (1b WT) and 1b Δ8 (FIG. 2). Furthermore, upon introduction of the most commonly identified resistance mutation, S282T, similar inhibitory patterns for either the 1b WT or the 1b Δ8 construct were noted for these same compounds (PSI-352666, IC₅₀=9-14 μM, a ˜2.5-fold increase in IC₅₀; 2′-C-MeGTP IC₅₀=>100 μM; a >10-fold increase)²² (FIG. 3).

The JFH1 isolate of genotype 2a is the only cloned HCV strain capable of efficient replication in cell culture as well as in vivo²³. A construct of 2a JFH1 NS5B with the β-hairpin loop replaced with a Gly-Gly linker (“2a Δ8”) was observed to be >100-fold more active than wild-type 2a in de novo RNA synthesis assays (FIG. 10b), capable of binding RNA in thermofluor analysis (FIGS. 6 and 7) and resulted in a similar IC₅₀ value for chain termination with PSI-352666 (6 μM) relative to wild-type 2a (FIG. 10b). Crystal structures of 2a Δ8 were obtained as described in Example 4.

### Example 4

### Crystallization and Structure Determination

Nearly 100 crystal structures of HCV NS5B have been reported covering genotypes 1a, 1b, 2a and 2b, although all structures lack the C-terminal membrane anchoring tail⁴. HCV NS5B exhibits the “right hand” shape common to many polymerases along with easily recognized “fingers,” “palm” and “thumb” domains⁸⁻¹⁰ (FIG. 10a). Extensive efforts to obtain a high resolution crystal structure of wild-type HCV polymerase in complex with growing RNA primer-template pairs have proven unsuccessful, although a structure has been reported with a polyuridine template in an unproductive conformation¹¹.

Insights from more recent RNA-bound complexes of RdRps from Norwalk virus¹⁷, poliovirus¹⁸, and foot and mouth disease virus (FMDV)¹⁹ which lack an equivalent β-hairpin loop prompted synthesis of an HCV NS5B 1b BK construct in which residues 442-456 had been excised. While this 1b Δ14 polymerase had reduced activity (data not shown), it provided the foundation for interest in replacing residues in the β-hairpin loop. (A report noted that replacement of residues 444-453 (Δ8) in this β-hairpin loop with a Tyr-Gly linker in HCV NS5B genotype 1b resulted in a 17-fold increase in primer extension activity over wild-type and the ability to bind primer-template RNA^{20,21}). An HCV NS5B 1b BK construct, 1b Δ8, in which residues 444-453 in the β-hairpin loop were substituted with a Gly-Gly linker was synthesized. A >10-fold increase in de novo RNA synthesis for 1b Δ8 (Example 3) was observed, as well as evidence for RNA binding via thermofluor analysis (Example 3). The JFH1 isolate of genotype 2a is the only cloned HCV strain capable of efficient replication in cell culture as well as in vivo²³. HCV polymerase genotype 2a JFH-1 constructs 2aWT and 2aΔ8, a construct of 2a JFH1 NS5B with the β-hairpin loop replaced with a Gly-Gly linker, were designed and synthesized, as described in Example 1. Polymerase construct 2a Δ8 was observed to be >100-fold more active than wild-type 2a, 2aWT, in de novo RNA synthesis assays (Example 3), capable of binding RNA in thermofluor analysis (Example 3), and resulted in a similar IC₅₀ value for chain termination with PSI-352666 (6 μM) relative to wild-type 2a (Example 3). As both the 1bΔ8 and 2aΔ8 polymerase constructs exhibited activity similar to the respective WT construct, an attempt was made to obtain crystal structures of the 1bΔ8 and 2aΔ8 polymerase constructs in order to demonstrate the structural basis for primer-template recognition and elongation.

### Methods

Crystallization conditions for the proteins were screened through a Hampton screen (available from hamptonresearch.com) and the JCSG+ (Emerald BioSystems). Crystals were grown using the sitting-drop vapor diffusion method in 96-well format Compact Junior crystallization plates from Emerald BioSystems using 0.4 μL of protein solution and an equal volume of precipitant equilibrated against 80 μL of precipitant at 16° C. Rod-shaped crystals (20×20×120 μm³) appeared within 3-5 days in several conditions from the JCSG+(Emerald BioSystems) and the Index (Hampton Research) sparse matrix screens.

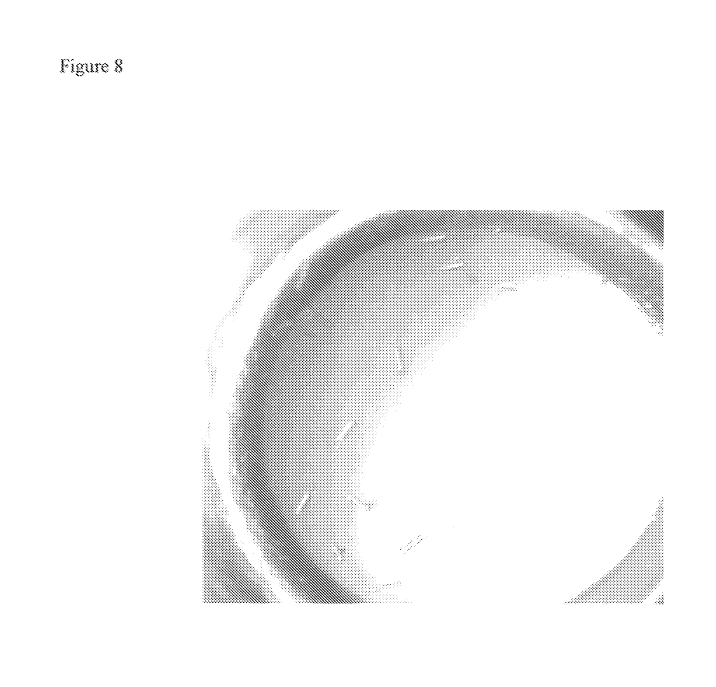

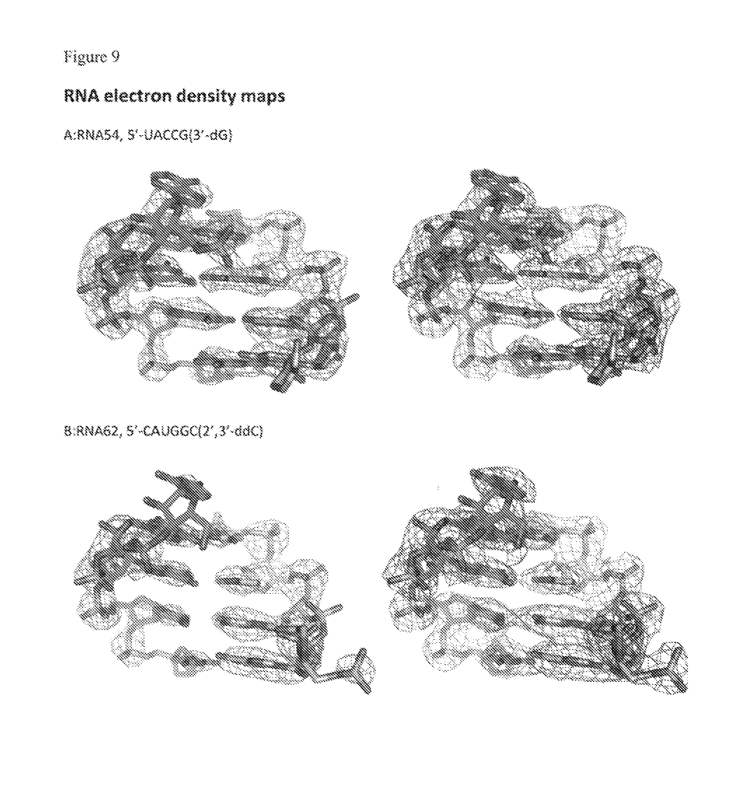

The apo NS5B 2a Δ8 structure was obtained from a crystal grown in the presence of 30% pentaerithritol ethoxylate, 0.1 M BisTris pH 6.5 and 50 mM ammonium sulfate (FIG. 8). Apo NS5B 2a Δ8 crystals grown in the presence of 25% PEG 3350, 0.1 M BisTris pH 5.5-6.5 and 0.2 M ammonium acetate (Index G6-G7) were soaked overnight at 16° C. with 0.2 mM 5′UACCG(3′ dG) or 5′-CAUGGC(ddC) (Dharmacon), precipitant and 15% ethylene glycol as cryoprotectant (FIG. 9). NS5B 2a Δ8 crystals grown or soaked in the presence of MES buffer were incompatible with RNA binding.

Crystals were harvested and flash frozen in liquid nitrogen for cryo-crystallography. The apo data set was collected at the Advanced Light Source (ALS 5.0.3) and the RNA-bound data sets were collected at the Advanced Photon Source (APS LS-CAT 21-ID-G). The data were reduced in XDS/XSCALE²⁹. The structures were solved by molecular replacement in PHASER³¹ using a previously determined apo structure of HCV NS5B 2a triple variant (to be published), which in turn was solved by molecular replacement using the wild-type HCV NS5B 2a structure (PDB ID 2XXD). The final models were produced after numerous reiterative rounds of refinement in REFMAC5³² and manual model building in COOT³³. Structures were assessed for correctness and validated using Molprobity³⁴.

### Results

Although the biological characterization of 1bΔ8 in Example 3 demonstrated functionality similar to 1bWT, a crystal structure of apo 1bΔ8 or of the ternary complex was not able to be obtained. A 2.5 Å resolution apo crystal structure of 2a Δ8 was obtained and coordinates for the crystal structure are shown in Table 1.

The obtained crystal structure revealed substantial structural changes relative to previously determined 2a NS5B structures²³⁻²⁵ with an overall r.m.s.d value of 1.8 Å. The 2a Δ8 Gly444-Gly445 linker was ordered and Phe551 was the last ordered residue. Alignment of the palm and fingers domains of a closed apo 2a structure²³ with the apo 2a Δ8 structure showed an overall ˜20° movement of the thumb domain (FIG. 11a). The lack of the β-hairpin loop, the disorder of the C-terminal linker region, and the movement of the thumb domain combined to generate a large cavity in the center of the polymerase.

The thumb domain movement was accompanied by significant reordering of residues 397-412 which connect the primer grip helix with the primer buttress helix (FIG. 11). In particular, Ile405, which was previously detailed to be important for de novo initiation across all genotypes²³, moved more than 12 Å away from the β-hairpin loop in the closed wild-type structure to extend the primer buttress helix and pack on top of the highly conserved Trp408 (FIG. 2). Trp408 stacked on top of the nearly invariant Phe429 in the closed wild-type structure, and both residues adopt different rotamer conformations in the 2aΔ8 structure.

In addition, the highly conserved Pro404, which contacts His95 of the finger domain in the closed apo structure (FIG. 11b), formed a key turn in the loop while packing on top of the main chain of Trp397 in the apo 2aΔ8 structure (FIG. 11c). This loop reordering may be critical to the transition from de novo initiation with GTP to elongation of the growing primer-template RNA. Comparison of the apo 2aΔ8 structure with other RdRp ternary complexes¹⁷⁻¹⁹ suggested that in this open conformation, HCV NS5B may be able to bind primer-template RNA.

Crystals of apo 2aΔ8 were soaked with symmetrical primer-template RNA pairs RNA54 and RNA 62 from Example 2 and structures were determined at 2.9 Å and 3.0 Å resolution (FIG. 12). The coordinates for the RNA-bound structures with RNA54 or RNA62 are shown in Table 2 and 3, respectively. Crystallographic statistics for apo 2aΔ8 and RNA-bound structures with RNA54 or RNA62 are shown in Table 7.

TABLE 7
Crystallographic statistics for HCV NS5B 2a JFH1 Δ8 apo and RNA-bound structures

| RNA | Apo | | 5′-UACCG(3′-dG) | | 5′-CAUGGC(2′,3′-ddC) | |
|---|---|---|---|---|---|---|
| Data collection | | | | | | |
| Beamline | ALS 5.0.3 | | APS 21-ID-D | | APS 21-ID-D | |
| Collection date | 11 Nov. 2011 | | 16 Dec. 2011 | | 16 Dec. 2011 | |
| Wavelength | 0.9765 | | 0.97856 | | 0.97856 | |
| Data reduction | | | | | | |
| Space Group | P6₅ | | P6₅ | | P6₅ | |
| Unit Cell | a = b = 140.69 Å, c = 92.63 Å | | a = b = 143.27 Å, c = 92.19 | | a = b = 142.73 Å, c = 91.50 | |
| | α = β = 90°, γ = 120° | | Å, α = β = 90°, γ = 120° | | Å, α = β = 90°, γ = 120° | |
| Solvent content (%) | 70.4 | | 71.0 | | 70.6 | |
| Vₘ (Å³/Da) | 4.15 | | 3.90 | | 3.84 | |
| Resolution (Å) | 50-2.5 | (2.57-2.50)^{a} | 50-2.9 Å | (2.98-2.90) | 50-3.0 Å | (3.08-3.00) |
| I/σ | 19.1 | (2.3) | 22.0 | (2.8) | 16.8 | (2.3) |
| Completeness (%) | 94.8 | (91.5) | 98.5 | (97.7) | 99.5 | (99.6) |
| R_{merge} | 0.078 | (0.675) | 0.092 | (0.863) | 0.099 | (0.745) |
| Multiplicity | 3.3 | (3.4) | 5.9 | (6.1) | 5.3 | (5.3) |
| Reflections | 34,324 | (2437) | 23,671 | (1738) | 21,289 | (1572) |
| Mosaicity | 0.2 | | 0.2 | | 0.4 | |
| Refinement | | | | | | |
| R | 0.215 | (0.411) | 0.200 | (0.364) | 0.197 | (0.329) |
| R_{free} | 0.257 | (0.463) | 0.242 | (0.421) | 0.241 | (0.372) |
| r.m.s.d. bonds (Å) | 0.012 | | 0.009 | | 0.009 | |
| r.m.s.d. angles (°) | 1.654 | | 1.336 | | 1.340 | |
| Mean B-factors (Å²) | 45.4 | | 46.0 | | 46.3 | |
| Validation | | | | | | |
| Ramachandran Favored (%) | 96.3 | | 94.8 | | 93.3 | |
| Ramachandran Allowed (%) | 100 | | 99.8 | | 99.6 | |
| Molprobity Score | 2.35 | (84^{th} percentile) | 2.50 | (93^{rd} percentile) | 2.50 | (94^{th} percentile) |

^{a}Values in parenthesis indicate the highest resolution shell. 20 shells were used in XSCALE.

A-form RNA was readily apparent in the resulting electron density maps (FIG. 12d and FIG. 9), clearly showing the differences in purine/pyrimidine pairings of the two RNA sequences. Both symmetrical primer-template RNA pairs were designed as obligate chain terminators with either a 3′-dG or a 2′,3′-ddC, and thus, unsurprisingly, a product state, pretranslocation registry was observed in both complexes (FIG. 12c). None of the nucleobase hydrogen bond acceptors or donors is recognized by the polymerase, indicating sequence independent recognition by the polymerase. The nucleobase of the pairing nucleotide of the template strand (residue+1 by convention) stacks on top of the strictly conserved Ile160, as predicted⁹, while the sugar stacks on top of Tyr162, which is conserved as Tyr or Phe (FIG. 13a). When the pairing nucleotide is a pyrimidine, residue+1 of the primer strand (equivalent to the incoming NTP) also packs with Ile160 (FIG. 13a), possibly accounting for some of the differences in purine/pyrimidine analog triphosphate inhibitor activity.

All of the phosphates and 2′-hydroxyls of the template strand are recognized by NS5B (FIG. 13b), demonstrating the importance of an RNA template for HCV. Likewise, the phosphates of the primer strand are recognized by Arg158 of the finger domain and the primer grip helix of the thumb domain, while the primer buttress helix forms van der Waals interactions with several primer strand sugars. The 2′-hydroxyl of primer residue+1 of the product, pretranslocation state, which resides at the same position as the incoming NTP in the substrate registry, is recognized by the side chain of Asp225 (FIG. 13c). As both structures contain a 3′-deoxy terminal residue, the other carboxylate oxygen of Asp225 is free to hydrogen bond with Asn291. The equivalent residue (Asp238) of the poliovirus RdRp was shown to adopt different conformations depending on the incoming NTP, translocation state, and presence of divalent metal ions¹⁸. None of the other 2′-hydroxyls of the primer strand are recognized by NS5B, consistent with reduced activity with DNA primers²⁶. Despite low sequence identity outside the catalytic residues, the overall primer-template RNA recognition strategy of HCV is essentially identical to that observed for Norwalk virus¹⁷, poliovirus¹⁸, and FMDV¹⁹ (FIG. 13d).

### Discussion

These structures demonstrate a molecular basis for primer-template recognition and elongation by HCV polymerase and provide insight into the structural basis by which resistance-derived mutations permit the polymerase to continue to function while diminishing the impact of the inhibitor. The more open nature of the primer-template-bound complexes described in this example also offers a more complete glimpse into the mode of action for allosteric inhibitors. Thus, these structures provide a valuable crystallization platform for structure-guided drug design, in particular for nucleotide analog inhibitors or NNIs that target the ternary complex. The methodology of deleting elements of the β-hairpin loop to afford the primer-template-bound complex may prove similarly useful through iterative application to other viral RdRps with this structural feature.

### REFERENCES

1. Behrens, S. E., Tomei, L. & De Francesco, R. Identification and properties of the RNA-dependent RNA polymerase of hepatitis C virus. EMBO J 15, 12-22 (1996).
2. Powdrill, M. H., Bernatchez, J. A. & Gotte, M Inhibitors of the Hepatitis C Virus RNA-Dependent RNA Polymerase NS5B. Viruses 2, 2169-95 (2010).
3. Sofia, M. J., Chang, W., Furman, P. A., Mosley, R. T. & Ross, B. S, Nucleoside, Nucleotide and Non-Nucleoside Inhibitors of Hepatitis C Virus NS5B RNA-Dependent RNA-Polymerase. J Med Chem, accepted (2011).
4. Caillet-Saguy, C., Simister, P.C. & Bressanelli, S. An objective assessment of conformational variability in complexes of hepatitis C virus polymerase with non-nucleoside inhibitors. J Mol Biol 414, 370-84 (2011).
5. Lavanchy, D. The global burden of hepatitis C. Liver Int 29 Suppl 1, 74-81 (2009).
6. Penin, F., Dubuisson, J., Rey, F. A., Moradpour, D. & Pawlotsky, J. M. Structural biology of hepatitis C virus. Hepatology 39, 5-19 (2004).
7. Luo, G. et al. De novo initiation of RNA synthesis by the RNA-dependent RNA polymerase (NS5B) of hepatitis C virus. J Virol 74, 851-63 (2000).
8. Ago, H. et al. Crystal structure of the RNA-dependent RNA polymerase of hepatitis C virus. Structure 7, 1417-26 (1999).
9. Bressanelli, S. et al. Crystal structure of the RNA-dependent RNA polymerase of hepatitis C virus. Proc Natl Acad Sci USA 96, 13034-39 (1999).
10. Lesburg, C. A. et al. Crystal structure of the RNA-dependent RNA polymerase from hepatitis C virus reveals a fully encircled active site. Nat Struct Biol 6, 937-43 (1999).
11. O'Farrell, D., Trowbridge, R., Rowlands, D. & Jager, J. Substrate complexes of hepatitis C virus RNA polymerase (HC-J4): structural evidence for nucleotide import and de-novo initiation. J Mol Biol 326, 1025-35 (2003).
12. Huang, H., Chopra, R., Verdine, G. L. & Harrison, S. C. Structure of a covalently trapped catalytic complex of HIV-1 reverse transcriptase: implications for drug resistance. Science 282, 1669-75 (1998).
13. Butcher, S. J., Grimes, J. M., Makeyev, E. V., Bamford, D. H. & Stuart, D. I. A mechanism for initiating RNA-dependent RNA polymerization. Nature 410, 235-40 (2001).
14. Choi, K. H. et al. The structure of the RNA-dependent RNA polymerase from bovine viral diarrhea virus establishes the role of GTP in de novo initiation. Proc Natl Acad Sci USA 101, 4425-30 (2004).
15. Yap, T. L. et al. Crystal structure of the dengue virus RNA-dependent RNA polymerase catalytic domain at 1.85-angstrom resolution. J Virol 81, 4753-65 (2007).
16. Malet, H. et al. Crystal structure of the RNA polymerase domain of the West Nile virus non-structural protein 5. J Biol Chem 282, 10678-89 (2007).
17. Zamyatkin, D. F. et al. Structural insights into mechanisms of catalysis and inhibition in Norwalk virus polymerase. J Biol Chem 283, 7705-12 (2008).
18. Gong, P. & Peersen, O. B. Structural basis for active site closure by the poliovirus RNA-dependent RNA polymerase. Proc Natl Acad Sci USA 107, 22505-10 (2010).
19. Ferrer-Orta, C. et al. Structure of foot-and-mouth disease virus RNA-dependent RNA polymerase and its complex with a template-primer RNA. J Biol Chem 279, 47212-21 (2004).
20. Hong, Z. et al. A novel mechanism to ensure terminal initiation by hepatitis C virus NS5B polymerase. Virology 285, 6-11 (2001).
21. Maag, D., Castro, C., Hong, Z. & Cameron, C. E. Hepatitis C virus RNA-dependent RNA polymerase (NS5B) as a mediator of the antiviral activity of ribavirin. J Biol Chem 276, 46094-98 (2001).
22. Furman, P. A. et al. Activity and the metabolic activation pathway of the potent and selective hepatitis C virus pronucleotide inhibitor PSI-353661. Antiviral Res 91, 120-32 (2011).
23. Schmitt, M. et al. A comprehensive structure-function comparison of hepatitis C virus strain JFH1 and J6 polymerases reveals a key residue stimulating replication in cell culture across genotypes. J Virol 85, 2565-81 (2011).
24. Biswal, B. K. et al. Crystal structures of the RNA-dependent RNA polymerase genotype 2a of hepatitis C virus reveal two conformations and suggest mechanisms of inhibition by non-nucleoside inhibitors. J Biol Chem 280, 18202-10 (2005).
25. Simister, P. et al. Structural and functional analysis of hepatitis C virus strain JFH1 polymerase. J Virol 83, 11926-39 (2009).
26. Yamashita, T. et al. RNA-dependent RNA polymerase activity of the soluble recombinant hepatitis C virus NS5B protein truncated at the C-terminal region. J Biol Chem 273, 15479-86 (1998).
27. Lorimer, D. et al. Gene composer: database software for protein construct design, codon engineering, and gene synthesis. BMC Biotechnol 9, 36 (2009).
28. Murakami, E. et al. Mechanism of activation of beta-D-2′-deoxy-2′-fluoro-2′-c-methylcytidine and inhibition of hepatitis C virus NS5B RNA polymerase. Antimicro Agents Chemothe 51, 503-09 (2007).
29. Kabsch, W. Xds. Acta Crystallogr D Biol Crystallogr 66, 125-32 (2010).
30. Choi, K. H. & Rossmann, M. G. RNA-dependent RNA polymerases from Flaviviridae. Curr Opin Struct Biol 19, 746-51 (2009).
31. McCoy, A. J. et al. Phaser crystallographic software. J Appl Crystallogr 40, 658-74 (2007).
32. Murshudov, G. N., Vagin, A. A. & Dodson, E. J. Refinement of macromolecular structures by the maximum-likelihood method. Acta Crystallogr D Biol Crystallogr 53, 240-55 (1997).
33. Emsley, P. & Cowtan, K. Coot: model-building tools for molecular graphics. Acta Crystallogr D Biol Crystallogr 60, 2126-32 (2004).
34. Chen, V. B. et al. MolProbity: all-atom structure validation for macromolecular crystallography. Acta Crystallogr D Biol Crystallogr 66, 12-21 (2010).
35. DeLano, W. L. The PyMOL molecular graphics system, (DeLano Scientific, San Carlos, Calif., 2002).
36. Crowther, G. J. et al. Use of thermal melt curves to assess the quality of enzyme preparations. Anal Biochem 399, 268-75 (2010).
37. Matulis, D., Kranz, J. K., Salemme, F. R. & Todd, M. J. Thermodynamic stability of carbonic anhydrase: measurements of binding affinity and stoichiometry using ThermoFluor. Biochemistry 44, 5258-66 (2005).
38. Bressanelli et al., Crystal Structure of the RNA-Dependent RNA Polymerase of Hepatitis C Virus. Proc. Natl. Acad. Sci. U.S.A., 1996, 96, 13035-39.

Lengthy table referenced here
US20150087045A1-20150326-T00001
Please refer to the end of the specification for access instructions.

Lengthy table referenced here
US20150087045A1-20150326-T00002
Please refer to the end of the specification for access instructions.

Lengthy table referenced here
US20150087045A1-20150326-T00003
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20150087045A1). An electronic copy of the table will also be available from the USPTO upon request and payment of the fee set forth in 37 CFR 1.19(b)(3).

1. An isolated polynucleotide comprising a nucleic acid encoding a polypeptide having at least 95% amino acid sequence identity to the amino acid sequence of SEQ ID NO: 8, wherein the polypeptide, as compared to the wild-type hepatitis C virus (HCV) genotype 2a NS5B RNA polymerase, has a deletion or substitution of a β hairpin corresponding to amino acid residues 442-454 of SEQ ID NO: 1 and has increased RNA synthesis activity.

2. The isolated polynucleotide of claim 1, wherein the polypeptide has a sequence of SEQ ID NO:8.

3. An isolated polypeptide expressed by the polynucleotide of claim 1.

4-8. (canceled)

9. A crystal comprising the polypeptide of claim 3.

10. The crystal of claim 9 further comprising RNA template primer molecules.

11-16. (canceled)

17. The crystal of claim 9 having the three dimensional coordinates of Table 1.

18. The crystal of claim 9 having the three dimensional coordinates of Table 2 or Table 3.

19-29. (canceled)


SEQUENCE LISTING (36 sequences)

SEQ ID NO: 1 - protein, 591 aa, Hepatitis C virus
SMSYSWTGALITPCSPEEEKLPINPLSNSLLRYHNKVYCTTSKSASQRAKKVTFDRTQVLDAHYDSVLKDIKLAASKVSARLLTLEEACQLTPPHSARSKYGFGAKEVRSLSGRAVNHIKSVWKDLLEDPQTPIPTTIMAKNEVFCVDPAKGGKKPARLIVYPDLGVRVCEKMALYDITQKLPQAVMGASYGFQYSPAQRVEYLLKAWAEKKDPMGFSYDTRCFDSTVTERDIRTEESIYQACSLPEEARTAIHSLTERLYVGGPMFNSKGQTCGYRRCRASGVLTTSMGNTITCYVKALAACKAAGIVAPTMLVCGDDLVVISESQGTEEDERNLRAFTEAMTRYSAPPGDPPRPEYDLELITSCSSNVSVALGPRGRRRYYLTRDPTTPLARAAWETVRHSPINSWLGNIIQYAPTIWVRMVLMTHFFSILMVQDTLDQNLNFEMYGSVYSVNPLDLPAIIERLHGLDAFSMHTYSHHELTRVASALRKLGAPPLRVWKSRARAVRASLISRGGKAAVCGRYLFNWAVKTKLKLTPLPEARLLDLSSWFTVGAGGGDIFHSVSRARPRSLLFGLLLLFVGVGLFLLPAR

SEQ ID NO: 2 - protein, 591 aa, Hepatitis C virus
SMSYTWTGALITPCAAEESKLPINALSNSLLRHHNMVYATTSRSAGLRQKKVTFDRLQVLDDHYRDVLKEMKAKASTVKAKLLSVEEACKLTPPHSAKSKFGYGAKDVRNLSSKAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASRLDLSGWFVAGYSGGDIYHSLSRARPRWFMLCLLLLSVGVGIYLLPNR

SEQ ID NO: 3 - protein, 577 aa, Artificial Sequence
  2a WT  [HCV NS5b 2a (1-565, WT, E86Q, E87Q)]His-6
MSSMSYSWTGALITPCSPEEEKLPINPLSNSLLRYHNKVYCTTSKSASQRAKKVTFDRTQVLDAHYDSVLKLAASKVSARLLTLQQACQLTPPHSARSKYGFGAKEVRSLSGRAVNHIKSVWKDLLEDPQTPIPTTIMAKNEVFCVDPAKGGKKPARLIVYPDLGVRVCEKMALYDITQKLPQAVMGASYGFQYSPAQRVEYLLKAWAEKKDPMGFSYDTRCFDSTVTERDIRTEESIYQACSLPEEARTAIHSLTERLYVGGPMFNSKGQTCGYRRCRASGVLTTSMGNTITCYVKALAACKAAGIVAPTMLVCGDDLVVISESQGTEEDERNLRAFTEAMTRYSAPPGDPPRPEYDLELITSCSSNVSVALGPRGRRRYYLTRDPTTPLARAAWETVRHSPINSWLGNIIQYAPTIWVRMVLMTHFFSILMVQDTLDQNLNFEMYGSVYSVNPLDLPAIIERLHGLDAFSMHTYSHHELTRVASALRKLGAPPLRVWKSRARAVRASLISRGGKAAVCGRYLFNWAVKTKLKLTPLPEARLLDLSSWFTVGAGGGDIFHSVSRARPRLEHHHHHH

SEQ ID NO: 4 - protein, 579 aa, Artificial Sequence
  1b WT  His-6[HCV NS5b 1B (1-570, L47Q, E86Q, E87Q, K114R, C-term delta21)]
MASHHHHHHSMSYTWTGALITPCAAEESKLPINALSNSLLRHHNMVYATTSRSAGQRQKKVTFDRLQVLDDHYRDVLKEMKAKASTVKAKLLSVQQACKLTPPHSAKSKFGYGAKDVRNLSSRAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPR

SEQ ID NO: 5 - protein, 579 aa, Artificial Sequence
  1b S282T  His-6[HCV NS5b 1B (1-570, L47Q, E86Q, E87Q, K114R, S282T, C-term delta21)]
MASHHHHHHSMSYTWTGALITPCAAEESKLPINALSNSLLRHHNMVYATTSRSAGQRQKKVTFDRLQVLDDHYRDVLKEMKAKASTVKAKLLSVQQACKLTPPHSAKSKFGYGAKDVRNLSSRAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRATGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPR

SEQ ID NO: 6 - protein, 571 aa, Artificial Sequence
  1b-delta8  His-6[HCV NS5b 1B (BK, QM, delta444-453, GG, C-term delta21)]
MASHHHHHHSMSYTWTGALITPCAAEESKLPINALSNSLLRHHNMVYATTSRSAGQRQKKVTFDRLQVLDDHYRDVLKEMKAKASTVKAKLLSVQQACKLTPPHSAKSKFGYGAKDVRNLSSRAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALGGIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPR

SEQ ID NO: 7 - protein, 571 aa, Artificial Sequence
  1b-delta8 S282T  His-6[HCV NS5b 1B (BK, QM, delta444-453, GG, C-term delta21)]
MASHHHHHHSMSYTWTGALITPCAAEESKLPINALSNSLLRHHNMVYATTSRSAGQRQKKVTFDRLQVLDDHYRDVLKEMKAKASTVKAKLLSVQQACKLTPPHSAKSKFGYGAKDVRNLSSRAVNHIHSVWKDLLEDTVTPIDTTIMAKNEVFCVQPEKGGRKPARLIVFPDLGVRVCEKMALYDVVSTLPQVVMGSSYGFQYSPGQRVEFLVNTWKSKKNPMGFSYDTRCFDSTVTENDIRVEESIYQCCDLAPEARQAIKSLTERLYIGGPLTNSKGQNCGYRRCRATGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALGGIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPR

SEQ ID NO: 8 - protein, 569 aa, Artificial Sequence
  2a-delta8  [HCV NS5b 2a (1-565, WT, E86Q, E87Q, delta8)]His-6
MSSMSYSWTGALITPCSPEEEKLPINPLSNSLLRYHNKVYCTTSKSASQRAKKVTFDRTQVLDAHYDSVLKLAASKVSARLLTLQQACQLTPPHSARSKYGFGAKEVRSLSGRAVNHIKSVWKDLLEDPQTPIPTTIMAKNEVFCVDPAKGGKKPARLIVYPDLGVRVCEKMALYDITQKLPQAVMGASYGFQYSPAQRVEYLLKAWAEKKDPMGFSYDTRCFDSTVTERDIRTEESIYQACSLPEEARTAIHSLTERLYVGGPMFNSKGQTCGYRRCRASGVLTTSMGNTITCYVKALAACKAAGIVAPTMLVCGDDLVVISESQGTEEDERNLRAFTEAMTRYSAPPGDPPRPEYDLELITSCSSNVSVALGPRGRRRYYLTRDPTTPLARAAWETVRHSPINSWLGNIIQYAPTIWVRMVLMTHFFSILMVQDTLDQNLGGVNPLDLPAIIERLHGLDAFSMHTYSHHELTRVASALRKLGAPPLRVWKSRARAVRASLISRGGKAAVCGRYLFNWAVKTKLKLTPLPEARLLDLSSWFTVGAGGGDIFHSVSRARPRLEHHHHHH

SEQ ID NO: 9 - RNA, 6 nt, Artificial Sequence
  Symmetrical primer-template RNA construct; RNA54; n = 3′-dG
uaccgn

SEQ ID NO: 10 - RNA, 7 nt, Artificial Sequence
  Symmetrical primer-template RNA construct; RNA62; n = 2′,3′-ddC
cauggcn

SEQ ID NO: 11 - RNA, 8 nt, Artificial Sequence
  Symmetrical primer-template RNA construct; RNA27
uacccggg

SEQ ID NO: 12 - RNA, 8 nt, Artificial Sequence
  Symmetrical primer-template RNA construct; RNA30; n = 3′-dG
uacccggn

SEQ ID NO: 13 - RNA, 10 nt, Artificial Sequence
  Symmetrical primer-template RNA construct; RNA29
uaccccgggg

SEQ ID NO: 14 - RNA, 10 nt, Artificial Sequence
  Symmetrical primer-template RNA construct; RNA32; n = 3′-dG
uaccccgggn

SEQ ID NO: 15 - RNA, 13 nt, Artificial Sequence
  Fused primer-template hairpin RNA construct; RNA35 (13-mer; 3 bp)
gagcccgaaaggg

SEQ ID NO: 16 - RNA, 17 nt, Artificial Sequence
  Fused primer-template hairpin RNA construct; RNA37 (17-mer; 5 bp)
gagcccccgaaaggggg

SEQ ID NO: 17 - RNA, 19 nt, Artificial Sequence
  Fused primer-template hairpin RNA construct; RNA38 (19-mer; 6 bp)
gagccccccgaaagggggg

SEQ ID NO: 18 - RNA, 21 nt, Artificial Sequence
  Fused primer-template hairpin RNA construct; RNA39 (21-mer; 7 bp)
gagcccccccgaaaggggggg

SEQ ID NO: 19 - RNA, 25 nt, Artificial Sequence
  Fused primer-template hairpin RNA construct; RNA41 (25-mer; 9 bp)
gagcccccccccgaaaggggggggg

SEQ ID NO: 20 - protein, 531 aa, Hepatitis C virus
  misc_feature (2)..(2): Xaa can be any naturally occurring amino acid
SXSYTWTGALITPCAAEESKLPINALSNSLLRHHNXVYATTSRSAGLRQKKVTFDRLQVLDDHYRDVLKEXKAKASTVKAKLLSVEEACKLTPPHSAKSKFGYGAKDVRNLSSKAVNHIHSVWKDLLEDTVTPIDTTIXAKNEVFCVQPEKGGRKPARLIVFPDLGVRVCEKXALYDVVSTLPQVVXGSSYGFQYSPGQRVEFLVNTWKSKKNPXGFSYDTRCFDSTVTENDIRVEESIYQCCDLAPEARQAIKSLTERLYIGGPLTNSKGQNCGYRRCRASGVLTTSCGNTLTCYLKASAACRAAKLQDCTXLVNGDDLVVICESAGTQEDAASLRVFTEAXTRYSAPPGDPPQPEYDLELITSCSSNVSVAHDASGKRVYYLTRDPTTPLARAAWETARHTPVNSWLGNIIXYAPTLWARXILXTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVK

SEQ ID NO: 21 - protein, 591 aa, Hepatitis C virus
SMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVTHINSVWKDLLEDNVTPIDTTIMAKNEVFCVQPEKGGRKPARLIVFPDLGVRVCEKMALYDVVTKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLARGGRAAICGKYLFNWAVRTKLKLTPIAAAGQLDLSGWFTAGYSGGDIYHSVSHARPRWIWFCLLLLAAGVGIYLLPNR

SEQ ID NO: 22 - protein, 591 aa, Hepatitis C virus
SMSYTWTGALITPCAAEESKLPINPLSNSLLRHHNMVYATTSRSASLRQKKVTFDRLQVLDDHYRDVLKEMKAKASTVKAKLLSIEEACKLTPPHSAKSKFGYGAKDVRNLSSRAVNHIRSVWEDLLEDTETPIDTTIMAKSEVFCVQPEKGGRKPARLIVFPDLGVRVCEKMALYDVVSTLPQAVMGSSYGFQYSPKQRVEFLVNTWKSKKCPMGFSYDTRCFDSTVTESDIRVEESIYQCCDLAPEARQAIRSLTERLYIGGPLTNSKGQNCGYRRCRASGVLTTSCGNTLTCYLKATAACRAAKLQDCTMLVNGDDLVVICESAGTQEDAAALRAFTEAMTRYSAPPGDPPQPEYDLELITSCSSNVSVAHDASGKRVYYLTRDPTTPLARAAWETARHTPINSWLGNIIMYAPTLWARMILMTHFFSILLAQEQLEKALDCQIYGACYSIEPLDLPQIIERLHGLSAFTLHSYSPGEINRVASCLRKLGVPPLRTWRHRARSVRAKLLSQGGRAATCGRYLFNWAVRTKLKLTPIPAASQLDLSGWFVAGYSGGDIYHSLSRARPRWFPLCLLLLSVGVGIYLLPNR

SEQ ID NO: 23 - protein, 591 aa, Hepatitis C virus
SMSYSWTGALITPCAAEETKLPINALSNSLLRHHNLVYSTTSRSAGQRQKKVTFDRLQVLDDHYRDVLKEAKAKASTVKAKLLSVEEACSLTPPHSARSKFGYGAKDVRSHSSKAIRHINSVWQDLLEDNTTPIDTTIMAKNEVFCVKPEKGGRKPARLIVYPDLGVRVCEKRALYDVVKQLPIAVMGTSYGFQYSPAQRVDFLLNAWKSKKNPMGFSYDTRCFDSTVTEADIRTEEDLYQSCDLVPEARAAIRSLTERLYIGGPLTNSKGQNCGYRRCRASGVLTTSCGNTITCYLKASAACRAAKLRDCTMLVCGDDLVVICESAGVQEDAANLRAFTEAMTRYSAPPGDPPQPEYDLELITSCSSNVSVAHDGAGKRVYYLTRDPETPLARAAWETARHTPVNSWLGNIIMFAPTLWVRMVLMTHFFSILIAQEHLEKALDCEIYGAVHSVQPLDLPEIIQRLHGLSAFSLHSYSPGEINRVAACLRKLGVPPLRAWRHRARSVRATLLSQGGRAAICGKYLFNWAVKTKLKLTPLPSASQLDLSNWFTGGYSGGDIYHSVSHVRPRWFFWCLLLLSVGVGIYLLPNR

SEQ ID NO: 24 - protein, 591 aa, Hepatitis C virus
SMSYSWTGALITPCSPEEEKLPINPLSNSLLRYHNKVYCTTSKSASLRAKKVTFDRMQALDAHYDSVLKDIKLAASKVTARLLTLEEACQLTPPHSARSKYGFGAKEVRSLSGRAVNHIKSVWKDLLEDTQTPIPTTIMAKNEVFCVDPTKGGKKAARLIVYPDLGVRVCEKMALYDITQKLPQAVMGASYGFQYSPAQRVEFLLKAWAEKKDPMGFSYDTRCFDSTVTERDIRTEESIYRACSLPEEAHTAIHSLTERLYVGGPMFNSKGQTCGYRRCRASGVLTTSMGNTITCYVKALAACKAAGIIAPTMLVCGDDLVVISESQGTEEDERNLRAFTEAMTRYSAPPGDPPRPEYDLELITSCSSNVSVALGPQGRRRYYLTRDPTTPIARAAWETVRHSPVNSWLGNIIQYAPTIWARMVLMTHFFSILMAQDTLDQNLNFEMYGAVYSVSPLDLPAIIERLHGLDAFSLHTYTPHELTRVASALRKLGAPPLRAWKSRARAVRASLISRGGRAAVCGRYLFNWAVKTKLKLTPLPEARLLDLSSWFTVGAGGGDIYHSVSRARPRLLLLGLLLLFVGVGLFLLPAR

SEQ ID NO: 25 - protein, 538 aa, Hepatitis C virus
SSMSYSWTGALITPCSPEEEKLPINPLSNSLLRYHNKVYCTTSKSASQRAKKVTFDRTQVLDAHYDSVLKDIKLAASKVSARLLTLQQACQLTPPHSARSKYGFGAKEVRSLSGRAVNHIKSVWKDLLEDPQTPIPTTIMAKNEVFCVDPAKGGKKPARLIVYPDLGVRVCEKMALYDITQKLPQAVMGASYGFQYSPAQRVEYLLKAWAEKKDPMGFSYDTRCFDSTVTERDIRTEESIYQACSLPEEARTAIHSLTERLYVGGPMFNSKGQTCGYRRCRASGVLTTSMGNTITCYVKALAACKAAGIVAPTMLVCGDDLVVISESQGTEEDERNLRAFTEAMTRYSAPPGDPPRPEYDLELITSCSSNVSVALGPRGRRRYYLTRDPTTPLARAAWETVRHSPINSWLGNIIQYAPTIWVRMVLMTHFFSILMVQDTLDQNLGGVNPLDLPAIIERLHGLDAFSMHTYSHHELTRVASALRKLGAPPLRVWKSRARAVRASLISRGGKAAVCGRYLFNWAVKTKLKLTPLPEARLL

SEQ ID NO: 26 - protein, 591 aa, Hepatitis C virus
SMSYSWTGALITPCGPEEEKLPISPLSNSLMRFHNKVYSTTSRSASLRAKKVTFDRVQVLDAHYDSVLQDVKRAASKVSARLLSVEEACALTPPHSAKSRYGFGAKEVRSLSRGAVNHIRSVWEDLLEDQHTPIDTTAMAKNEVFCIDPAKGGKKPARLIVYPDLGVRVCEKMALYDIAQKLPKAIMGPSYGFQYSPAERVDFLLKAWGSKKDPMGFSYDTRCFDSTVTERDIRTEESIYQACSLPQEARTVIHSITERLYVGGPMTNSKGQSCGYRRCRASGVFTTSMGNTMTCYIKALAACKAAGIVDPTMLVCGDDLVVISESQGNEEDERNLRAFTEAMTRYSAPPGDLPRPEYDLELITSCSSNVSVALDSRGRRRYFLTRDPTTPITRAAWETVRHSPVNSWLGNIIQYAPTIWVRMVIMTHFFSILLAQDTLNQNLNFEMYGAVYSVNPLDLPAIIERLHGLDAFSLHTYSPHELSRVAATLRKLGAPPLRAWKSRARAVRASLIIQGGRAATCGRYLFNWAVKTKLKLTPLPEASRLDLSGWFTVGAGGGDIFHSVSHARPRLLLLCLLLLSVGVGIFLLPAR

SEQ ID NO: 27 - protein, 591 aa, Hepatitis C virus
SMSYSWTGALITPCSPEEEKLPINPLSNSLLRYHNKVYCTTSRSASQRAKKVTFDRVQLLDSHYESVLKDVKQAATKVSAKLLSIEEACALTPPHSARSKYGFGAKEVRSLSRRAVDHIKSVWEDLLEDHCSPIDTTIMAKNEVFCVDPTKGGKKPARLIVYPDLGVRVCEKMALYDITQKLPVAVMGQSYGFQYSPAQRVDFLLQAWKEKKTPMGFSYDTRCFDSTVTERDIRTEESIYLSCSLPEEARTAIHSLTERLYVGGPMTNSKGQSCGYRRCRASGVLTTSMGNTLTCYVKAKAACNAAGIVAPTMLVCGDDLVVISESQGVEEDERNLRVFTEAMTRYSAPPGDPPKAEYDLELITSCSSNVSVALDPRGRRRYYLTRDPTTPLARAAWETARHSPVNSWLGNIIQYAPTVWVRMVLMTHFFSVLMAQDTLDQDLNFEMYGAVYSVSPLDLPAIIERLHGLEAFSLHSYSPHELTRVAAALRKLGAPPLRAWKSRARAVRASLISRGGSAATCGRYLFNWAVRTKLKLTPLPAARLLDLSSWFTVSAGGGDIYHSVSRARPRLLLLGLLLLCVGVGIFLLPAR

SEQ ID NO: 28 - protein, 591 aa, Hepatitis C virus
SMSYSWTGALITPCGPEEEKLPINPLSNSLLRYHNKVYSTTSRSASQRAKKVTFDRVQLLDSHYDQVLKDIKLAASKVSANLLSIEEACALTPPHSARSKYGFGAKEVRSLSRKAVDHIKSVWKDLLEDQQTPIPTTIMAKNEVFCIDPTKGGKKAARLIVFPDLGVRVCEKMALYDITQKLPQAVMGASYGFQYSPAQRVDFLLRAWKEKKDPMGFSYDTRCFDSTVTERDIRTEESIYLACSLPEEARVAIHSLTERLYVGGPMMNSKGQSCGYRRCRASGVLTTSMGNTITCYVKALAACKAAGIVAPTMLVCGDDLVVISESQGAEEDERNLRVFTEAMTRYSAPPGDPPKPEYDLELITSCSSNVSVALDQHGRRMYYLTRDPSTPLARAAWETARHSPVNSWLGNIIQYAPTIWVRMVLMTHFFSVLMAQETLDQDLNFEMYGAVYSVNPLDLPAIIERLHGLEAFSLHGYSPTELTRVAAALRKLGAPPLRAWKSRARAVRASLISQGGRAATCGFYLFNWAVRTKRKLTPLPAARRLDLSGWFTVGAGGGDIYHSVSRARPRFLLLCLLLLSVGVGIFLLPAR

SEQ ID NO: 29 - protein, 591 aa, Hepatitis C virus
SMSYSWTGALITPCSAEEEKLPISPLSNSLLRHHNLVYSTSSRSASQRQKKVTFDRLQVLDDHYKTALKEVKERASRVKARMLTIEEACALVPPHSARSKFGYSAKDVRSLSSRAINQIRSVWEDLLEDTTTPIPTTIMAKNEVFCVDPAKGGRKPARLIVYPDLGVRVCEKRALYDVIQKLSIETMGPAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAANLRNPDFLVCGDDLVVVAESDGVDEDRAALRAFTEAMTRYSAPPGDAPQATYDLELITSCSSNVSVARDDKGRRYYYLTRDATTPLARAAWETARHTPVNSWLGNIIMYAPTIWVRMVMMTHFFSILQSQEILDRPLDFEMYGATYSVTPLDLPAIIERLHGLSAFTLHSYSPVELNRVAGTLRKLGCPPLRAWRHRARAVRAKLIAQGGKAKICGLYLFNWAVRTKTNLTPLPAAGQLDLSSWFTVGVGGNDIYHSVSRARTRHLLLCLLLLTVGVGIFLLPAR

SEQ ID NO: 30 - protein, 591 aa, Hepatitis C virus
SMSYSWTGAIITPCSAEEEKLPISPLSNSLLRHHNLVYSTSSRSAAARQKKVTFDRLQVLDDHYKNVLKEVKERASGVKGRLLSFEEACSLVPPHSGRSKYGYSAKDVRSLSSKAMNQIRSVWEDLLEDNSTPIPTTIMAKNEVFSVNPAKGGRKPARLIVYPDLGVRVCEKRALYDVIQKLSIATMGPAYGFQYSPKQRVEHLLKMWTSKKTPLGFSYDTRCFDSTVTEHDIRTEEGIYQCCDLEPEARKAISALTERLYIGGPMYNSKGLQCGYRRCRASGVLPTSFGNTITCYIKATAASRAAGLKNPSFLVCGDDLVVISESCGVEEDRTALRAFTEAMTRYSAPPGDAPQPTYDLELISSCSSNVSVACDGAGKRYYYLTRDPETPLARAAWETARHTPVNSWLGNIIMFAPTIWVRMVLITHFFSILQAQEQLERALDFEMYGATYSVTPLDLPAIIERLHGLSAFSLHGYSPTELNRVAGALRKLGIPPLRAWRHRARAVRAKLIAQGGKARICGLYLFNWAVRTKTKLTPLPTAGQLDLSSWFTVGVGGNDIYHSVSRARTRHLLLCLLLLTVGVGIFLLPAR

SEQ ID NO: 31 - protein, 591 aa, Hepatitis C virus
SMSYSWTGALITPCAAEEEKLPISPLSNSLLRHHNLVYSTSSRSAAQRQKKVTFDRLQVLDDHYNTTLKEIKELASGVKAELLSVEEACRLVPSHSARSKFGYGAKEVRSLSSKAINHINSVWEDLLEDNTTPIPTTIMAKNEVFAVAPHKGGRKPARLIVYPDLGVRICEKRALYDVIQKLPSAIMGSAYGFQYSPKQRVEYLLKMWNSKKTPLGFSYDTRCFDSTVTEQDIRVEESIYQACDLKDEARRVITSLTERLYCGGPMFNSKGQHCGYRRCRASGVLPTSFGNTVTCYLKAKAATKAAGIKDPSFLVCGDDLVVIAESAGIDEDKSALRAFTEAMTRYSAPPGDPPQPTYDLELITSCSSNVSVAHDGAGKRYYYLTRDPETPLARAAWETARHTPVNSWLGNIIMYAPTIWVRMVIMTHFFSILQAQEQLEKALDFEMYGAVYSVTPLDLPAIIERLHGLSAFSLHSYSPVELNRVAGALRKLGIPPLRAWRHRARAVRAKLISQGGKAKICGLYLFNWAVRTKAKLTPLPQAGLLDLSRWFTVGAGGNDIYHSVSRARSRHLLLGLLLLTVGVGIFLLPAR

SEQ ID NO: 32 - protein, 591 aa, Hepatitis C virus
SMSYSWTGALVTPCAAEESKLPISPLSNSLLRHHNMVYATTTRSAVTRQKKVTFDRLQVVDSTYNEVLKEIKARASRVKPRLLTTEEACDLTPPHSARSKFGYGKKDVRSHSRKAINHISSVWKDLLDDNNTPIPTTIMAKNEVFAVNPAKGGRKPARLIVYPDLGSRVCEKRALHDVIKKTALAVMGAAYGFQYSPAQRVEFLLTAWKSKNDPMGFSYDTRCFDSTVTEKDIRVEEEVYQCCDLEPEARKVITALTDRLYVGGPMHNSKGDLCGYRRCRATGVYTTSFGNTLTCYLKATAAIRAAALRDCTMLVCGDDLVVIAESDGVEEDNRALRAFTEAMTRYSAPPGDAPQPAYDLELITSCSSNVSVAHDVTGKKVYYLTRDPETPLARAVWETVRHTPVNSWLGNIIVYAPTIWVRMILMTHFFSILQSQEALEKALDFDMYGVTYSITPLDLPAIIQRLHGLSAFTLHGYSPHELNRVAGALRKLGVPPLRAWRHRARAVRAKLIAQGGRAKICGIYLFNWAVKTKLKLTPLPAAAKLDLSGWFTVGAGGGDIYHSMSHARPRYLLLCLLILTVGVGIFLLPAR

SEQ ID NO: 33 - protein, 591 aa, Hepatitis C virus
SMSYTWTGALITPCSAEEEKLPINPLSNTLLRHHNLVYSTSSRSAGLRQKKVTFDRLQVLDDHYREVVDEMKRLASKVKARLLPLEEACGLTPPHSARSKYGYGAKEVRSLDKKALKHIEGVWQDLLDDSDTPLPTTIMAKNEVFAVEPSKGGKKPARLIVYPDLGVRVCEKRALYDVAQKLPTALMGPSYGFQYSPAQRVDFLLKAWKSKKIPMAFSYDTRCFDSTITEHDIMTEESIYQSCDLQPEARVAIRSLTQRLYCGGPMYNSKGQQCGYRRCRASGVFTTSMGNTMTCYIKALASCRAAKLRDCTLLVCGDDLVAICESQGTHEDEASLRAFTEAMTRYSAPPGDPPVPAYDLELVTSCSSNVSVARDASGNRIYYLTRDPQVPLAKAAWETAKHSPVNSWLGNIIMYAPTLWARIVLMTHFFSVLQSQEQLEKTLAFEMYGSVYSVTPLDLPAIIQRLHGLSAFSLHSYSPSEINRVASCLRKLGVPPLRAWRHRARAVRAKLIAQGGRAAICGIYLFNWAVKTKRKLTPLADADRLDLSSWFTVGAGGGDIYHSMSRARPRNLLLCLLLLSVGVGIFLLPAR

SEQ ID NO: 34 - protein, 591 aa, Hepatitis C virus
SMSYSWTGALITPCAAEEEKLPINPLSNSLIRHHNMVYSTTSRSASLRQKKVTFDRLQVFDQHYQDVLKEIKLRASTVQARLLSIEEACDLTPSHSARSKYGYGAQDVRSHASKAINHIRSVWEDLLEDSDTPIPTTIMAKNEVFCVDPSKGGRKPARLIVYPDLGVRVCEKMALYDVTRKLPQAVMGSAYGFQYSPNQRVEYLLKMWRSKKVPMGFSYDTRCFDSTVTERDIRTENDIYQSCQLDPVARRAVSSLTERLYVGGPMVNSKGQSCGYRRCRASGVLPTSMGNTLTCYLKAQAACRAANIKDCDMLVCGDDLVVICESAGVQEDTASLRAFTDAMTRYSAPPGDVPQPTYDLELITSCSSNVSVAHDGNGKRYYYLTRDCTTPLARAAWETARHTPVNSWLGNIIMFAPTIWVRMVLMTHFFSILQSQEQLEKALDFDIYGVTYSVSPLDLPAIIQRLHGMAAFSLHGYSPTELNRVGACLRKLGVPPLRAWRHRARAVRAKLIAQGGKAAICGKYLFNWAVKTKLKLTPLVSASKLDLSGWFVAGYDGGDIYHSVSQARPRLLLLGLLLLTVGVGIFLVPAR

SEQ ID NO: 35 - protein, 591 aa, Hepatitis C virus
SMSYTWTGALITPCAAEEEKLPINPLSNSLIRHHNMVYSTTSRSAGLRQKKVTFDRLQVVDQHYQDVLKEIKLRASTVHARLLSTEEACSLTPPHSARSRYGYGARDVRSHTSKAVKHIDSVWEDLLEDNATPIPTTIMAKNEVFCVDPSKGGRKPARLIVYPDLSVRVCEKMALYDVTQKLPKTVMGSAYGFQYSPSQRVEYLLKMWRSKKTPMGFSYDTRCFDSTVTERDIRTEEDIYQSCQLDPTARKAISSLTERLYCGGPMFNSKGESCGYRRCRASGVLTTSLGNTLTCYLKAQAACRAANIKNFDMLVCGDDLVVICESAGVQEDVVALRAFTDAMIRYSAPPGDAPQPTYDLELITSCSSNVSVAHDGTGQRYYYLTRDCTTPLARAAWETARHTPVNSWLGNIIMYAPTIWVRMVLMTHFFSILQCQEQLEAALNFDMYGVTYSVTPLDLPAIIQRLHGMAAFSLHGYSPTELNRVGASLRKLGAPPLRAWRHRARAVRAKLIAQGGKAAICGKYLFNWAVKTKLKLTPLAAASQLDLSGWFVAGYDGGDIYHSVSRARPRLLLLGLLLLTVGVGIFLLPAR

SEQ ID NO: 36 - protein, 591 aa, Hepatitis C virus
SMSYSWTGALITPCAAEEEKLPISPLSNALIRHHNLVYSTTSRSASLRQKKVTFDRVQVVDQHYYDVLKEIKTKASGVSAKLLSVEEACALTPPHSARSKFGYGAKEVRGLASKAVNHINSVWEDLLEDNSTPIPTTIMAKNEVFCVDAQKGGRKPARLIVYPDLGVRVCEKRALYDVTQKLPIAVMGAAYGFQYSPKQRVDYLLKMWRSKKTPMGFSYDTRCFDSTVTERDIRTEEDIYQCCQLDPVAKKAITSLTERLYCGGPMYNSRGQSCGYRRCRASGVLTTSLGNTLTCYLKAQAACRAAKLKDFDMLVCGDDLVVISESMGVAEDASALRAFTEAMTRYSAPPGDDPQPEYDLELITSCSSNVSVAHDGAGQRYYYLTRDPLTPLSRAAWETARHTPVNSWLGNIIMYAPTIWVRMVLMTHFFAILQSQEILHKALDFDMYGVTYSVTPLDLPYIIQRLHGMAAFSLHGYSPGELNRVASCLRKLGAPPLRAWRHRARAVRAKLIAQGGKHAICGKYLFNWAVRTKLKLTPLRGAANLDLSGWFVSGGSGGDIFHSVSRARPRNLLLCLLLLTVGVGIFLLPAR